(19) United States
(12) Patent Application Publication
(10) Pub. No.: US 2023/0210747 A1
(43) Pub. Date: Jul. 6, 2023

(54) Anti-Ageing Cosmetic Compositions for Men and Women, Containing Bioactive Protein, and the Method of its Production

(71) Applicant: REELABS PVT. LTD., Mumbai (IN)

(72) Inventors: Abhijit Bopardikar, Mumbai (IN); Oleksandr Kukharchuk, Mumbai (IN); Padma Priya Anand Baskaran, Mumbai (IN); Andrii Kukharchuk, Mumbai (IN); Sunil Pophale, Mumbai (IN); Rohit Kulkarni, Mumbai (IN)

(21) Appl. No.: 17/802,174

(22) PCT Filed: Feb. 25, 2021
(86) PCT No.: PCT/IB2021/051574

§ 371 Date: Aug. 25, 2022

(30) Foreign Application Priority Data
Feb. 26, 2020 (IN) 202021008114

(51) Int. Cl. A61K 8/64; A61Q 19/08

(52) U.S. Cl. CPC A61K 8/64; A61Q 19/08

(57) ABSTRACT

The present invention relates to anti-aging composition comprising bioactive protein obtained by cultivation of placenta, method of preparing the composition and use as a cosmetic composition.

### FIELD OF THE INVENTION

This invention can be used in cosmetology for the production of skin anti-aging creams and hair lotions that improve the quality of hair and prevent hair loss and the appearance of gray hair.

### BACKGROUND OF THE INVENTION

Ageing is the result of the biological accumulation of damage in our cells. Hearing loss, poor eyesight, skin wrinkles, hair loss, weak muscles, slow heart rate, high blood pressure, decreased mental agility are the general symptoms of ageing. The factors that affect the ageing process are high frequency radiation, low frequency radiation, free radicals, nutrition, excessive or insufficient sunlight, insufficient sleep, excessive sexual activity, poisons, insufficient or improper or excessive exercise.

Throughout life, some cells ageing and die by the mechanism of programmed cell death (apoptosis), and their place is taken by other cells that are formed from stem cells. This process is called physiological regeneration. Stem cells are undifferentiated cells, which have the potential of differentiating into a various cell type. Stem cells are distinguished from other cell types by two important characteristics. First, they are unspecialized cells capable of renewing themselves through cell division, sometimes after long periods of inactivity. Further, under certain physiologic or experimental conditions, they can be induced to become tissue-specific or organ-specific cells with special functions. In some organs, such as the gut and bone marrow, stem cells regularly divide to repair and replace worn out or damaged tissues. In other organs, however, such as the pancreas and the heart, stem cells only divide under special conditions.

Stem cells receive a regulatory signal for division and differentiation into specialized cells from the stem cell niche. A stem-cell niche is an area of a tissue that provides a specific microenvironment, in which stem cells are present in an undifferentiated and self-renewable state. Cells of the stem-cell niche interact with the stem cells to maintain them or promote their differentiation. A Nature Insight review defines niche as follows: “Stem-cell populations are established in “niches”-specific anatomic locations that regulate how they participate in tissue regeneration, maintenance and repair. The niche saves stem cells from depletion, while protecting the host from over-exuberant stem-cell proliferation. It constitutes a basic unit of tissue physiology, integrating signals that mediate the balanced response of stem cells to the needs of organisms. Yet the niche may also induce pathologies by imposing aberrant function on stem cells or other targets. The interplay between stem cells and their niche creates the dynamic system necessary for sustaining tissues, and for the ultimate design of stem-cell therapeutics. . . . The simple location of stem cells is not sufficient to define a niche. The niche must have both anatomic and functional dimensions.”

During embryonic development, various niche factors act on embryonic stem cells to alter gene expression, and induce their proliferation or differentiation for the development of the fetus. Within the human body, stem-cell niches maintain adult stem cells in a quiescent state, but after tissue injury, the surrounding micro-environment actively signals to stem cells to promote either self-renewal or differentiation to form new tissues. Several factors are important to regulate stem-cell characteristics within the niche: cell-cell interactions between stem cells, as well as interactions between stem cells and neighbouring differentiated cells, interactions between stem cells and adhesion molecules, extracellular matrix components, the oxygen tension, growth factors, cytokines, and the physicochemical nature of the environment including the pH, ionic strength and metabolites, like ATP, are also important. The stem cells and niche may induce each other during development and reciprocally signal to maintain each other during adulthood.

Adult stem cells remain in an undifferentiated state throughout adult life. However, when they are cultured in vitro, they often undergo an aging process in which their morphology is changed and their proliferative capacity is decreased. It is believed that correct culturing conditions of adult stem cells needs to be improved so that adult stem cells can maintain their stemness over time. Similar processes occur in vivo: when the niche (microenvironment) of stem cells ages, the regulatory signals for the division and differentiation of stem cells are disrupted, which, in turn, disrupts physiological regeneration.

As the Drosophila female ages, the stem cell niche undergoes age-dependent loss of germline stem cells (GSC) presence and activity. These losses are thought to be caused in part by degradation of the important signaling factors from the niche that maintains GSCs and their activity. Progressive decline in GSC activity contributes to the observed reduction in fecundity of Drosophila melanogaster at old age; this decline in GSC activity can be partially attributed to a reduction of signaling pathway activity in the GSC niche. It has been found that there is a reduction in Dpp and Gbb signaling through aging. In addition to a reduction in niche signaling pathway activity, GSCs age cell-autonomously. In addition to studying the decline of signals coming from the niche, GSCs age intrinsically; there is age-dependent reduction of adhesion of GSCs to the cap cells and there is accumulation of Reactive Oxygen species (ROS) resulting in cellular damage which contributes to GSC aging. There is an observed reduction in the number of cap cells and the physical attachment of GSCs to cap cells through aging. Shg is expressed at significantly lower levels in an old GSC niche in comparison to a young one.

Prolonged spermatogenesis relies on the maintenance of somatic stem cells (SSCs), however, this maintenance declines with age and leads to infertility. Mice between 12 and 14 months of age show decreased testis weight, reduced spermatogenesis and SSC content. Although stem cells are regarded as having the potential to infinitely replicate in vitro, factors provided by the niche are crucial in vivo. Indeed, serial transplantation of SSCs from male mice of different ages into young mice 3 months of age, whose endogenous spermatogenesis had been ablated, was used to estimate stem cell content given that each stem cell would generate a colony of spermatogenesis. The results of this experiment showed that transplanted SSCs could be maintained far longer than their replicative lifespan for their age. In addition, a study also showed that SSCs from young fertile mice could not be maintained nor undergo spermatogenesis when transplanted into testes of old, infertile mice. Together, these results points towards a deterioration of the SSC niche itself with aging rather than the loss of intrinsic factors in the SSC.

The epidermis is comprised of at least three major stem cell populations: the hair follicle bulge, the sebaceous gland, and the basal layer of interfollicular epithelium. Because these subpopulations are responsible for regulating epithelial stratification, hair folliculogenesis, and wound repair throughout life, the epidermis has become a model system to study regeneration. Elegant lineage tracing and gene mapping experiments have elucidated key programs in epidermal homeostasis. Specifically, components of the wingless-type (Wnt)/β-catenin, sonic hedgehog (Shh), and transforming growth factor (TGF)-β/bone morphogenetic protein (BMP) pathways appear to be particularly relevant to epidermal stem cell function. Microarray analyses have even indicated that hair follicle stem cells share some of the same transcriptomes as other tissue-specific stem cells, suggesting that conserved molecular machinery may control how environmental stimuli regulate the stem cell niche.

Epithelial stem cells from the bulge, sebaceous gland, and basal epithelium have common features, including expression of K5, K14, and p63, and their intimate association with an underlying basement membrane (BM). These cells reside in the basal layer of stratified epithelium and exit their niche during differentiation. This process is mediated in part by BM components such as laminin and cell surface transmembrane integrins that control cell polarity, anchorage, proliferation, survival, and motility. Epithelial progenitor cells are also characterized by elevated expression of E-cadherin in adherens junctions and reduced levels of desmosomes, underscoring the importance of both extracellular and intercellular cues in stem cell biology. In addition to complex intraepithelial networks, signals from the dermis (e.g., periodic expression of BMP2 and BMP4) are thought to regulate epithelial processes. Dermal-derived stem cells may even differentiate into functional epidermal melanocytes, suggesting that mesenchymal-epithelial transitions may underlie skin homeostasis, as has been shown in hepatic stem cells. Recently, it has been demonstrated that irreversibly committed progeny from an epithelial stem cell lineage may be “recycled” and contribute back to the regenerative niche, further highlighting the complexity of the epidermal regeneration.

In contrast to the highly cellular nature of the epidermis, the dermis is composed of a heterogeneous matrix of collagens, elastins, and glycosaminoglycans interspersed with cells of various embryonic origin. Recent studies suggest that a cell population within the dermal papilla of hair follicles may function as adult dermal stem cells. This dermal unit contains at least three unique populations of progenitor cells differentiated by the type of hair follicle produced and the expression of the transcription factor Sox2. Sox2-expressing cells are associated with Wnt, BMP, and fibroblast growth factor (FGF) signaling whereas Sox2-negative cells utilize Shh, insulin growth factor (IGF), Notch, and integrin pathways. Skin-derived precursor (SKP) cells have also been isolated from dermal papillae and can be differentiated into adipocytes, smooth myocytes, and neurons in vitro. These cells are thought to originate in part from the neural crest and have been shown to exit the dermal papilla niche and contribute to cutaneous repair.

Researchers have also demonstrated that perivascular sites in the dermis may act as an MSC-like niche in human scalp skin. These perivascular cells express both NG2 (a pericyte marker) and CD34 (an MSC and hematopoietic stem cell marker) and are predominantly located around hair follicles. Perivascular MSC-like cells have been shown to protect their local matrix microenvironment via tissue-inhibitor-of-metalloproteinase (TIMP-) mediated inhibition of matrix metalloproteinase (MMP) pathways, suggesting the importance of the extracellular matrix (ECM) niche in stem cell function. Interestingly, even fibroblasts have been shown to maintain multilineage potential in vitro and may play important roles in skin regeneration that have yet to be discovered.

The hair follicle stem cell niche is one of the more closely studied niches. The bulge area at the junction of arrector pili muscle to the hair follicle sheath has been shown to host the skin stem cells which can contribute to all epithelial skin layers. There cells are maintained by signaling in concert with niche cells—signals include paracrine (e.g. sonic hedgehog), autocrine and juxtacrine signals. The bulge region of the hair follicle relies on these signals to maintain the stemness of the cells. Fate mapping or cell lineage tracing has shown that Keratin 15 positive stem cells' progeny participate in all epithelial lineages. The follicle undergoes cyclic regeneration in which these stem cells migrate to various regions and differentiate into the appropriate epithelial cell type. Some important signals in the hair follicle stem cell niche produced by the mesenchymal dermal papilla or the bulge include BMP, TGF-β and Fibroblast growth factor (FGF) ligands and Wnt inhibitors. While, Wnt signaling pathways and β-catenin are important for stem cell maintenance, over-expression of β-catenin in hair follicles induces improper hair growth. Therefore, these signals such as Wnt inhibitors produced by surrounding cells are important to maintain and facilitate the stem cell niche.

The hair follicle is a regenerating organ where stem cells allow for this massive large-scale renewal. The hair follicle is composed of an outer root sheath, an inner root sheath, and the hair shaft. The proliferating undifferentiated matrix cells give rise to the inner root sheath and the hair shaft and are surrounded by a dermal papilla of specialized mesenchymal cells. The dermal papilla instructs the formation of the follicle, but the characteristics of the follicle are acquired by epithelial information. The lower portion of the follicle goes through a growth cycle that involves the phases of anagen (active growth), catagen (destruction) and telogen (quiescence). These different phases last for varying time periods depending on the hair follicle location and function. The matrix cells proliferate rapidly during the anagen phase, migrate upwards then differentiate into the cell types of the inner root sheath and hair shaft. During the catagen phase, the lower follicle undergoes apoptotic death and the dermal papilla moves upwards until it reaches the area beneath the bulge. It remains there during telogen. Once the dermal papilla recruits stem cells from the bulge, anagen begins a new and the follicle can regenerate through proliferation and differentiation.

Scientists are studying the various components of the niche and trying to replicate the in vivo niche conditions in vitro. This is because for regenerative therapies, cell proliferation and differentiation must be controlled in flasks or plates, so that sufficient quantity of the proper cell type are produced prior to being introduced back into the patient for therapy. On the other hand, under aging conditions, when the regulatory signals of the skin and scalp stem cells niche are disturbed, it is possible to model such signals from the outside by applying creams and lotions containing the signal molecules of the niche, which are most important for activation and division of stem cells—cytokines and growth factors. This application can restore the processes of physiological regeneration of the skin and leads to its rejuvenation.

The human placenta is a unique temporary organ which ensures mutual coexistence of the organisms of mother and fetus, determining growth and development of the latter. The main functions of the placenta are ensuring the supply, growth, and development of the fetus, as well as removing metabolic products and preventing immune rejection. Since the placenta is a provisional organ, it becomes a salvage material after delivery. For decades, clinicians and researchers work on the application of the placenta for therapeutic purposes, initially in the form of extracts and cell or tissue transplants, thus accumulating substantial empirical experience. However, at the same time, a large amount of research was little systemized and not always correlated with conventional pharmaceutical and other methods of treatment. Recent developments of cell therapy approach along with opportunities for autobanking significantly increased the interest in the placenta as a source of biological material.

Due to a range of historical reasons, a significant amount of the studies is unfortunately not included into modern international databases, such as PubMed. For example, most of the works of academician Vladimir P. Filatov, founder of the Institute of Tissue Therapy and Eye Diseases (USSR), in the 1930s to 1960s were not even translated to English, highly limiting the audience of this valuable publications. At the same time, his fundamental work on the use of tissue therapy methods has been published in over 3000 scientific works; thousands of patients received effective treatment with devitalized placental medications; and dozens of departments and institutes on tissue therapy were established on this basis. Also, currently, more information on the centuries of experience of application of placental derivatives in traditional Chinese medicine becomes available from the recent publications.

The researchers have used in their studies the fragments of placental tissue, amniotic and chorionic membranes, umbilical cord, amniotic fluid, placental extracts and lyophilizates, and cord blood serum, as well as various types of differentiated cells and stem cells. Such material has been used in native form as well as after chemical and thermal processing, after cryopreservation and sublimation. Methods of application widely vary such as subcutaneous, intramuscular, intravenous, intraorperative, as biocovers, and substitutive material, as well as via oral administration. The views on the mechanism of action of placental preparations have been changing along with the development of biology and medicine. The researchers hypothesized and attempted to explain the patterns of therapeutic effects by vitamin, mineral, protein, and peptide composition, by the presence of “resistance factors,” “preservation factors,” cytokines, hormones, and stem cells. Such approach could explain common features of placental derivatives and shed light on the mechanism aspect and unique biological activity. At the same time, very often, the studies of the beginning of 21st century repeat the works from the mid-twentieth century, explaining obtained results in a different manner, while observing similar effects under the same pathological conditions.

High number of publications is related to the studies of placental extracts. Such extracts are obtained by lysing human placental tissues collected at full-term delivery. Therefore, the extracts do not contain cells but are rich in a wide range of proteins, minerals, amino acids, and steroid hormones. According to the data of various research groups, such extracts possess anti-inflammatory, analgesic, antioxidant, cyto- and radioprotective, and anti-allergic properties and express hormonal activity, as well as stimulate proliferation and reparative processes. Placental extracts were shown to enhance the proliferation of fibroblasts and cord blood cells in vitro. At the same time, it was noted that extracts isolated from the late gestation placenta possess the highest biological activity. Cytoprotective and antioxidant properties of the extracts are usually associated with the protein components; in particular, they are correlated with the concentration of alpha-fetoprotein. Animal model studies showed that prophylactic administration of the extracts increases the resistance of animals to oxidative stress. Placental extracts reduce the concentration of free radicals, inflammatory cytokines IL6, TNF, and IL1, at the same time increasing the colony formation of progenitor cells in vitro and reducing oxidative and radiation damage of the cells. Analysis of biosafety of placental extracts revealed the absence of toxic or mutagenic influence on cell cultures and adult animal models; however, fetotoxicity in animals at early gestation was reported.

Placental extracts have been applied for the treatment of a wide variety of pathological conditions—most commonly in surgery, neurology, gynecology, and dermatology. Pronounced positive effects were received in the treatment of wounds, nonhealing ulcers, and burns: rate of epithelialization was significantly increased and a decrease of infiltration and reduction of the pain syndrome were observed. The extracts accelerate the wound healing in animals with the diabetes model, which can be interpreted as a treatment for diabetic neuropathy and angiopathy. The mechanism of action of placental extracts in the wound healing is associated with the increase of TGF-β in the early phase of regeneration and VEGF in the late phase, as well as with the presence of FGF, amplification of angiogenesis, and the increase of expression of CD31. Application of placental extracts in menopausal disorders allowed reducing the number and severity of hot flushes, irritability, and normalize hormonal profile; the amount of estrogen receptors in the experiment was increased, and the effects of vaginal atrophy were reduced, while the activity of osteoblasts was improved. Experimental studies on the effect of placental extracts on behavior and physical condition in the animal model showed decrease in symptoms of fatigue and increased resistance to physical stress. This phenomenon was explained by the rise of the level of intracellular calcium, activation of splenocytes and T cells, and reduction of synthesis of proinflammatory cytokines associated with fatigue (IL6, TNF, and IFNγ). Similar results were obtained in preclinical studies.

Methods of chemical preservation, high-temperature sterilization, hypothermic storage, low-temperature storage, and cryosublimation have been used for the storage of placental components. Selection of the storage method depends on the type of material and the purpose of its further application. Methods of sterilization by filtration or autoclaving have been used for devitalization of the extracts containing peptides. Devitalization methods allow storing the material at the room temperature without additional equipment; however, the properties of such biological objects are significantly altered. For example, devitalization of amniotic membrane significantly reduces the immunogenicity and highly extends its biodegradation period.

Hypothermic and subnormothermic storage of biomaterial ensures preservation for a short period of time, required for delivery of the material to a laboratory or clinics with minor structural and biochemical injuries. The most conventional method for storage of biological objects, which provides high levels of preservation for a long period of time, is cryopreservation. Cord blood serum, placental extracts, cell suspensions, chorionic and amniotic membranes, and placental tissue are all suitable for cryopreservation procedures. Possibility to isolate MSCs with a stable genome from cryopreserved placental tissues, similar to the population of cells isolated from the native (fresh) tissues, significantly extends the scopes of cryopreservation of the placental material. The placenta is a unique object for low-temperature biobanking and autobanking. In most countries, the application of placenta does not face ethical issues and women positively evaluate this possibility, otherwise considering the material as a “waste.” Donation of the placenta is physiologically indifferent for the donor. At the same time, it provides a large amount of material suitable for direct application and for long-term storage in initial state or after processing, as well as for the preparation of extracts and isolation of cells or individual components. Biobanking technologies might offer a lifelong availability of the autologous placental material or tissue-engineered constructs, readily available for immediate application for a patient.

Considerable amount of information on the properties, experimental studies, and possibilities of clinical application of placental components is accumulated to date. The researchers showed the potential use of placental components in various fields of biology and medicine. However, many findings are not confirmed independently by different research groups and have not been performed on the amount of material sufficient enough for clinically significant statistical conclusions. Early works were primarily devoted to the study of placental extracts as hormonal agents and biostimulators, as well as amniotic membranes as biocovers. Cord blood cells are the most widely used placental component in modern medicine, being applied in the stem cell transplantation. Amniotic membrane is successfully applied in the ophthalmic practice, surgery, and wound healing. Novel technologies based on the application of placental MSCs and autobanking are considered as the most promising and prospective for the near future in the field of regenerative and reconstructive therapies as well as in bioengineering. The interest of researches in placental extracts and placental blood serum is still low, despite the widespread application of various fetal sera in the cell and tissue culture media.

Independent research groups at different time points received similar data on the properties and effects of application of the various components of the placenta for correction of a wide spectrum of pathologies. In most of their opinion, general therapeutic properties of various placental components are expressed in stimulation of reparative processes and anti-inflammatory and immunomodulatory effects. The mechanism of action of the various components of the placenta on the recipient's organism is associated with a shift from Th1 to Th2 type of immune response, suppression of the synthesis of IL6, TNF, and IFNγ, and enhancement of the synthesis of IL10, VEGF, and trophic factors.

Various research teams have verified the effects from the application of placenta and its derivatives on the nonhealing wounds, ulcers, disorders of the reproductive system (infertility and menopausal syndrome), autoimmune pathologies, and diabetes, as well as neurological disorders. The use of stem cell therapy methods is accepted as an addition rather than an alternative to conventional medical approaches, since it often does not cover all components of the pathogenesis of the targeted diseases. Considering the clinical potential and high perspectives of the placental material as an object for autobanking, it may be recommended to preserve not only the individual cell populations but also fragments of membranes, tissue, placental extracts, and cord blood serum.

There are no doubts in the conception of placental components as a rich source of biologically active substances and stem cells. However, in the studies carried out, predominantly the postpartum placenta was used, although the placenta of the first trimester of gestation contains much more unique biologically active proteins that directly affect organogenesis, including the development of skin and hair follicles.

Therefore, there is an unmet need in the art to provide a method for producing biologically active protein compositions from placental tissue at various stages of its early gestational development and their use in cosmetology for skin and hair rejuvenation; for the isolation of such compositions according to the present invention, it is necessary to have several placentas obtained as a result of medical termination of pregnancy at different stages of gestation, as well as a cosmetic base compatible with such compositions for the production of creams and lotions. Thus, natural compositions of biologically active proteins were found using the method of their extraction from the abortive placenta, excluding the homogenization of the amnion and chorion; extraction of growth factors and cytokines from explants of abortive placenta; creation of creams and lotions containing placental growth factors and cytokines of early placenta development; tested their stability when mixed with bases of face creams and hair lotions; and also tested their effectiveness in preventing aging of the skin of the face and hair.

### SUMMARY OF THE INVENTION

We have developed a technical protocol for production of anti-ageing cosmetic compositions for men and woman comprising bioactive protein, which are obtained by the method of cultivation of explants XX-placenta (for women's cosmetics) or XY-placenta (for men's cosmetics) comprises the steps of: Making explants of XX-placenta or XY-placenta in gestation term not less 11 weeks, and not more 12 weeks; Cultivation explants of XX-placenta or XY-placenta until the moment of migration and proliferation of cells on an area of no more than 4 cm² around each explant; Filtration of the conditioned medium of XX-placenta or XY-placenta explants and adding a proteolysis inhibitor to the medium; Control of biological safety of the conditioned medium of XX-placenta or XY-placenta explants (microbial, viral and fungal contamination); Controlling the concentration of biologically active proteins in a conditioned medium of XX-placenta or XY-placenta explants; Adding of XX- or XY-placental compositions of biologically active proteins to the ingredients of cosmetic products; Controlling the concentration of biologically active proteins of the XX-placenta or XY-placenta in ready-to-use cosmetic products.

Besides, we studied safety and efficacy of male skin cream, female skin cream, male hair lotion and female hair lotion, which contain biologically active proteins XX-placenta (women's cosmetics) or XY-placenta (men's cosmetics). Results of the study showed safety and high efficiency of biologically active proteins, which are produced by explants of the early XX- or XY-placenta.

### BRIEF DESCRIPTION OF THE DRAWINGS

FIG. 1. Abortive placenta, 12 weeks gestation, view from the umbilical cord and amnion.

FIG. 2. Abortive placenta, 12 weeks gestation, view from the chorion.

FIG. 3. Placental explants preparation. (A): placenta, gestational age 12 weeks. (B): Removal of blood clots. (C): Washing pieces of the placenta from blood impurities. (D): Transfer of pieces of placenta inside the flask for incubation in a CO₂ incubator. (E): Placental explants inside the flask.

FIG. 4. Migration of cells from the placenta and their growth in culture. (A):—cell growth around placental explants. (B, C): initiation of cell migration from placental explants and their proliferation. (D, E): Cell proliferation around placental explants reaches 4 cm².

FIG. 5. Differences of growth factors and cytokines concentrations in conditional media of XX- and XY-placenta explants. Remark: *—differences are statistically significant.

FIG. 6. Results of a study on the compatibility and stability of biologically active proteins of the XY-placenta in a male skin cream.

FIG. 7. Evaluation of the effectiveness of a male face cream containing biologically active peptides. The period of using the cream is 3 months. (A): Dynamics of change of the skin elasticity and firmness by a pinch test. (B): Example. In some case skin elasticity increase by 11%. (C): Dynamics of anti-sagging effect by VAS scale. (D, E): Dynamics of collagen fiber content. (F): Dynamics of the pigment content in the skin. (G, H): Example. In some case the skin pigment content showed a reduction of 27%.

FIG. 8. Continuation of the evaluation of the male face cream containing biologically active peptides effectiveness. The period of using the cream is 3 months. (A, B): Dynamics of change of the skin sebum content. (C): Example. In some cases, the skin sebum content decreased twice. (D): Dynamics of change of the skin moisture content. (E, F): Example. In some cases, the moisture content of the skin increased more when 4 times. (G): Dynamics of under eye puffiness by VAS scale. (H): Dynamics of Global Assessment Score.

FIG. 9. Results of a study on the compatibility and stability of biologically active proteins of the XX-placenta in a female skin cream.

FIG. 10. Evaluation of the effectiveness of a female face cream containing biologically active peptides. The period of using the cream is 3 months. (A): Dynamics of the skin the elasticity and firmness change by the pinch test. (B, C): Example. In some women the skin showed 20% improvement from the baseline to the end of the study. (D): Dynamics of the of anti-sagging effect by VAS score. (E): Dynamics of the collagen fiber content. (F, G): Example. In some women the collagen fiber content in the skin showed 24% increase from the baseline to the end of the study. (H): Dynamics of the skin pigment content. (I, J): Example. In some women the pigment content showed a reduction of 9% from the baseline to the end of the study.

FIG. 11. Continuation of the evaluation of the female face cream containing biologically active peptides effectiveness. The period of using the cream is 3 months. (A): Dynamics of the skin sebum content. (B, C): Example. In one case of initial high skin sebum content, this indicator decreased by 4%. (D, E): Example. In others case of initial high skin sebum content, the decrease in sebum content was more significant—by 8%. (F): Dynamics of the skin moisture content. (G): Example. In some cases, the moisture content of the skin increased almost doubled. (H): Dynamics of the under-eye puffiness by VAS scale. (I): Dynamics of the Global Assessment Score.

FIG. 12. Results of the study of compatibility and stability of biologically active proteins of XY-placenta in male hair lotion.

FIG. 13. Evaluation of the effectiveness of a male hair lotion containing biologically active peptides of XY placenta. The period of using the cream is 2 months. (A): Dynamics of the hair fall by comb's test. (B): Dynamics of the hair growth (hair/sq·cm). (C, D): Example. The new hair growth after completion of treatment. (E): Dynamics of the smoothening and softness of the hair. (F): Dynamics of the hair sheen. (G): Dynamics of the average number of hairs with split ends in 1 cm². (H): Dynamics of the hair strength by the pull test.

FIG. 14. Continuation of the evaluation of the male hair lotion containing biologically active peptides of XY placenta effectiveness. The period of using the cream is 2 months. (A): Dynamics of the hair density (hair/sq. inch). (B, C): Example. The increase in hair density (photos).

FIG. 15. Results of the study of compatibility and stability of biologically active proteins of XX-placenta in female hair lotion.

FIG. 16. Evaluation of the effectiveness of a female hair lotion containing biologically active peptides. The period of using the cream is 2 months. (A): Dynamics of the hair fall by comb's test. (B): Dynamics of the hair growth (hair/sq·cm). (C, D): Example. The new hair growth after completion of treatment. (E): Dynamics of the smoothening and softness of the hair. (F): Dynamics of the hair sheen. (G): Dynamics of the average number of hairs with split ends in 1 cm². (H): Dynamics of the hair strength by the pull test.

FIG. 17. Continuation of the evaluation of the female hair lotion containing biologically active peptides effectiveness. The period of using the cream is 2 months. (A): Dynamics of the hair density (hair/sq. inch). (B, C): Example. The increase in hair density (photos).

### DETAILED DESCRIPTION OF THE INVENTION

This invention is based on method for the preparation of human abortive XX- or XY-placenta; preparation of placental explants, including amnion and chorion; sowing explants on a nutrient medium; cultivating explants until the first wave of massive cell migration from placental explants appears; adding a proteolysis inhibitor to the nutrient medium; filtration of the conditioned medium; determination of the concentration of biologically active proteins (growth factors and cytokines) in a conditioned medium; control of contamination of the conditioned medium by microorganisms, viruses and fungi; adding a conditioned medium to the cosmetic base of skin rejuvenation creams and hair lotion; monitoring the stability of biologically active proteins in cosmetic bases and evaluating the cosmetic effectiveness of the resulting creams and lotions against skin and hair aging.

The main requirement for abortive XX- or XY-placenta is absence of bacteria, fungus and virus contamination. Biological safety of biological active proteins composition of abortive placenta is provided by stringent control in all stages of production—from procurement of anatomical material of abortive placenta which which was obtained as a result of medical termination of pregnancy till the preparation of anti-ageing creams and lotions for skin and hair.

Abortive XX- or XY-placenta dispatch (Requirements to Donors). Inclusion criteria for selection of donor: the donor should be 18 yrs of age or above; the donor should be free from all the infectious diseases viz: HIV-1 & 2, HBV, HCV, CMV, VDRL; the duration of pregnancy should be between 11 to 12 weeks; the donor should undergo medical termination of pregnancy (MTP) and give her consent for the same. Exclusion criteria for donor: age of the pregnant women is less than 18 years; absence of “The Informed Consent for HIV Test”, “The Informed Consent for MTP” or “The Informed Consent for the collection of the abortive placenta”, of the pregnant women; period of pregnancy is less 11 weeks or above 12 weeks; pregnant women is detected with infectious diseases including HIV 1/2, HCV, HBV, CMV, VDRL; known history for intrauterine fetal death; if the aborted material is collected as a result of spontaneous abortion; known for clear signs of congenital anomalies or infection in fetus.

Screening tests for donor of abortive XX- or XY-placenta. All the screening tests for the selection of donor were performed as per the “Guidelines for Stem Cell Research and Therapy (2007)” and “Guidelines for Stem Cell Research (2013)” jointly issued by DBT & ICMR (India). The donors were tested for the infectious diseases including HIV 1/2, HCV, HBV, CMV, and VDRL.

Medical termination of pregnancy (MTP) was performed by the recognized gynecologist at the Gynecology Department of Hospital. Only after the fulfillment of all the selection criteria, donors were allowed to fill “The Informed Consent form for MTP”. A separate consent form for MTP was provided by hospital. The “Informed consent form for donating the abortion placenta” was provided by Hospital, where the pre-abortion tests and MTP was carried out. Procuring abortive placenta tissue would be implemented only by obstetrician-gynecologist and nurses of the medical institutions in which abortion was performed.

Collection of abortive XX- or XY-placenta performed in sterile condition without changes in abortion technology, as permitted in India. Transportation of abortive placenta performed in special cryo-bags, which eliminates the possibilities of microbial contamination while transportation. Preliminary processing includes washing of abortive placenta from blood and washed out solution taken for emergency microbial contamination analysis by the method of express endotoxin analysis.

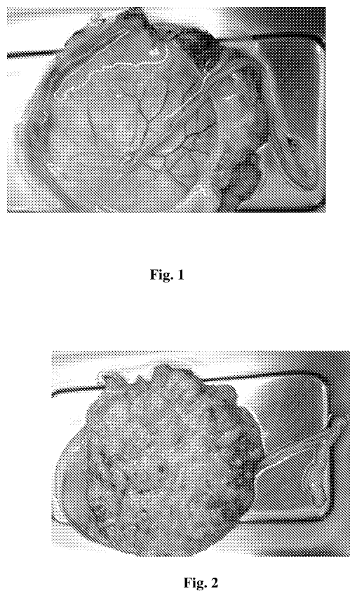

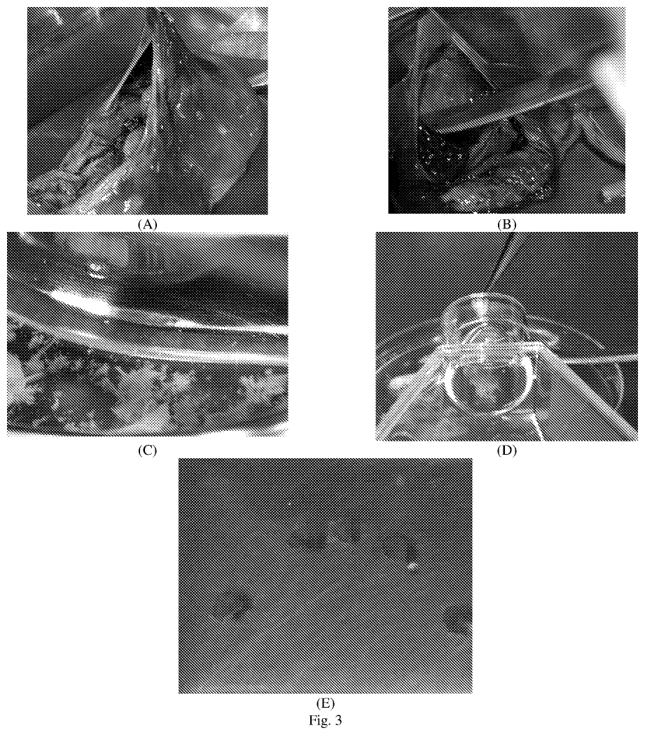

The choice of gestational age is due to the fact that organogenesis occurs at 10-11 weeks of intrauterine development. By this period, the laying of all definitive tissues and internal organs is completed. At 12 weeks, placenta formation ends (FIG. 1, FIG. 2), fetal thin skin is formed, and hair follicle formation is complete. It is believed that during this period of intrauterine development the placenta can produce biologically active proteins necessary for the further maturation of the skin and hair follicles.

After thorough washing of the placenta from blood (three times, chilled phosphate buffer) The separation of small pieces of tissue of the placenta (explants of the amnion and chorion) weighing 10 mg is performed mechanically using tweezers under sterile conditions. Then, 20 placental explants were placed in T75 flasks for cultivation in DMEM medium (10% FBS) for 4-5 days in a CO₂ incubator at 37° C. (FIG. 3A-E).

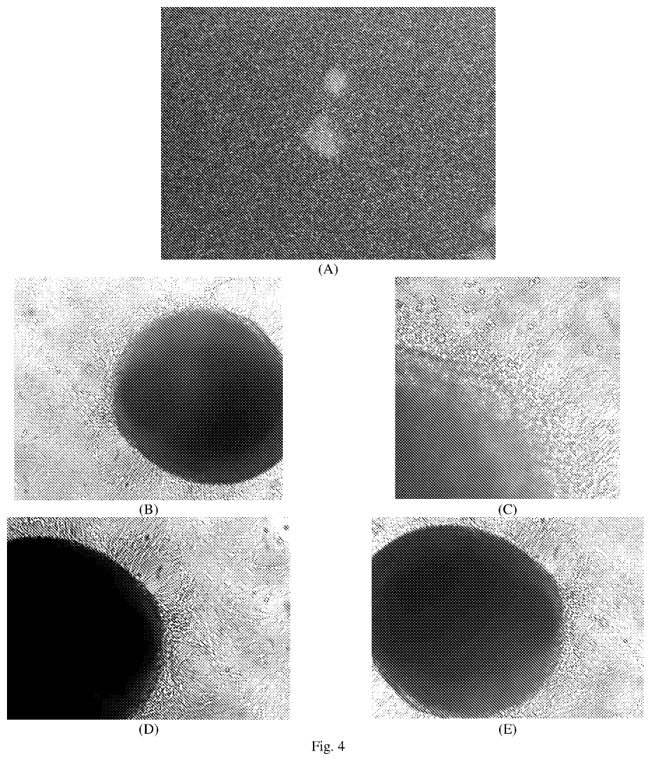

Control of cell migration from placental extracts and the onset of the first wave of their proliferation were performed daily (FIG. 4A-E). When the proliferation of cells around the explant reached 4 cm² (4-5 days of cultivation), the incubation was stopped. To prevent the degradation of biologically active proteins under the action of tissue proteolytic enzymes of the placental explants, a proteolysis inhibitor aprotenin was added to each flask at a final concentration of 200 KIU/ml. The conditioned medium was filtered through 40 μm filters, placed in 50.0 ml cryostable tubes and frozen in a refrigerator at minus 80° C.

Biosafety control. Part of the conditioned medium was sent to an independent lab for biosafety control (study for bacterial sterility, contamination of viruses, fungus and transmission infections: HIV1/HIV2, HbsAg, HCV, HBV, HSV 1/2, CMV, Treponema pallidum, Toxoplasma gondii, Micoplasma, Ureaplasma, Chlamidii, EBV by means of polymerase chain reaction).

Determination of the concentration of biologically active proteins in the conditioned medium was carried out by the ELISA method using commercial reagent kits from RayBiotech. Investigated concentrations of: Hepatocyte Growth Factor (HGF), Vascular Endothelial Growth Factor A (VEGF-A), Keratinocyte Growth Factor or Fibroblast Growth Factor 7 (FGF-7), Epidermal Growth Factor (EGF), Kit Ligand or Stem Cell Factor (SCF), Transforming Growth Factor beta 1 (TGFβ1), Tumor Necrosis Factor alpha (TNFα), Angiopoietin 1 (ANGPT1), Basic Fibroblast Growth Factor (bFGF), and Somatomedin C or Insulin-like Growth Factor 1 (IGF-1).

After cultivation of XY-placenta explants in a conditioned medium, the concentrations of the studied growth factors and cytokines were: EGF—14.04±2.12 pg/ml, FGF-7—187.05±56.66 pg/ml, HGF—1490.15±274.52 pg/ml, TNF-alpha— 430.46±45.25 pg/ml, IGF-1—778.91±74.64 ng/ml, VEGF-A—24.64±6.46 ng/ml, SCF—19.70±2.12 ng/ml, TGF-beta 1—122.21±27.78 ng/ml, ANGPT1—132.31±18.99 ng/ml, bFGF—77.27±9.70 ng/ml. After cultivation of XX-placenta explants in a conditioned medium, the concentrations of the studied growth factors and cytokines were: EGF—13.03±2.42 pg/ml, FGF-7—177.760±43.73 pg/ml, HGF—67.06±18.38 pg/ml, TNF-alpha— 442.99±51.61 pg/ml, IGF-1—597.52±41.92 ng/ml, VEGF-A—27.47±5.96 ng/ml, SCF—9.90±1.21 ng/ml, TGF-beta 1—430.46±59.89 ng/ml, ANGPT1—129.99±13.03 ng/ml, bFGF—28.64±7.17 ng/ml.

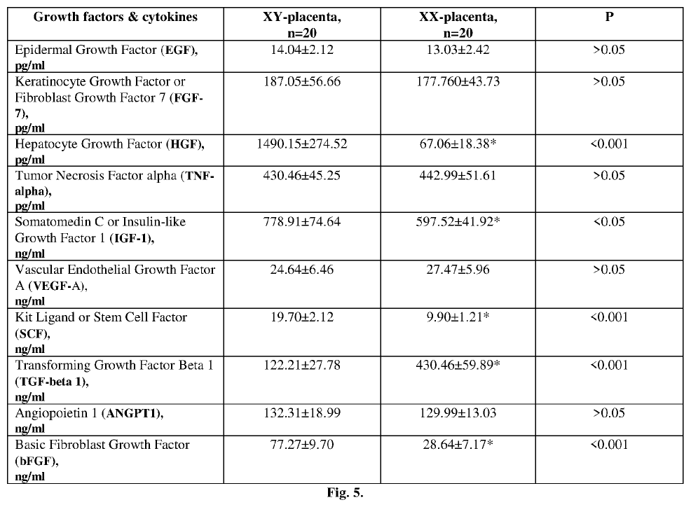

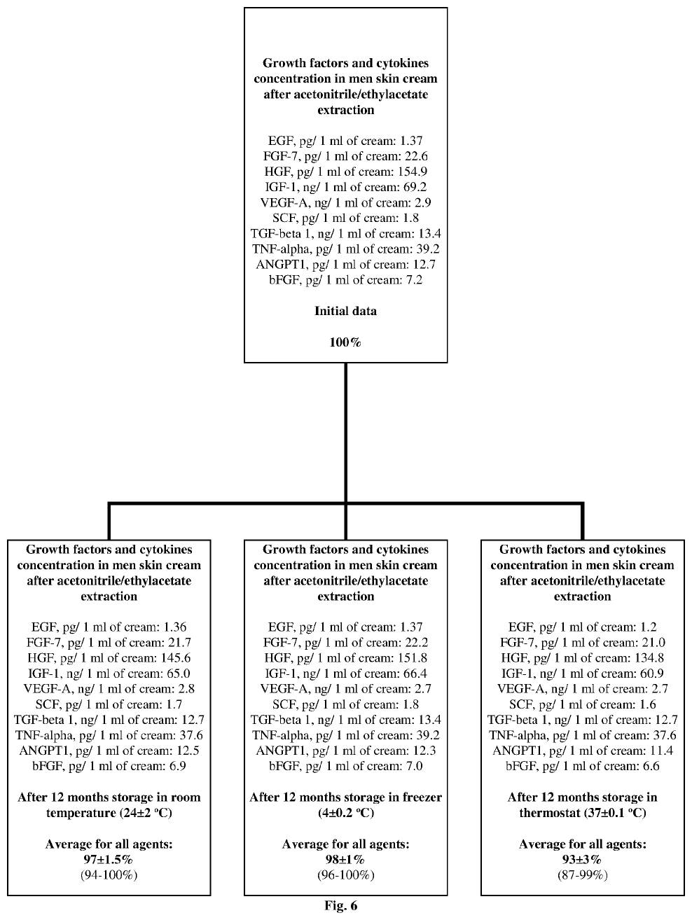

Thus, half of the 10 studied growth factors and cytokines in conditioned medium of XY- and XX-placenta explants differed significantly and authentically: XY-placental explants produced 22 times more HGF, 2 times more SCF, 1.3 times more IGF-1, and almost 3 times more bFGF than XX-placental explants. In turn, XX-placental explants produced 3.5 times more TGF-beta 1 than XY-placental explants (FIG. 5).

The cosmetic composition of biologically active proteins for men is produced using XY-placenta explants, and consists of a conditioned medium with such concentrations of growth factors and cytokines:

EGF—from 10 to 18 pg/ml
FGF-7—from 74 to 300 pg/ml
HGF—from 941 to 2039 pg/ml
TNF-alpha— from 340 to 521 pg/ml
IGF-1—from 630 to 928 ng/ml
VEGF-A—from 12 to 38 ng/ml
SCF—from 15.5 to 24 ng/ml
TGF-beta 1—from 66.5 to 178 ng/ml
ANGPT1—from 94 to 170 ng/ml
bFGF—from 58 to 97 ng/ml

The cosmetic composition of biologically active proteins for women is produced using XX-placenta explants, and consists of a conditioned medium with such concentrations of growth factors and cytokines:

EGF—from 8 to 18 pg/ml
FGF-7—from 90 to 265 pg/ml
HGF—from 30 to 104 pg/ml
TNF-alpha— from 340 to 546 pg/ml
IGF-1—from 514 to 681 ng/ml
VEGF-A—from 16 to 39 ng/ml
SCF—from 7.5 to 12 ng/ml
TGF-beta 1—from 311 to 550 ng/ml
ANGPT1—from 104 to 156 ng/ml
bFGF—from 14 to 43 ng/ml

The topical anti-ageing composition comprising bioactive protein according to present invention is meant to be applying on the skin, on the hair or scalp or on head. The topical anti-ageing composition comprising bioactive protein may be in the form of creams, lotions, serums, masks, balms, emulsions, microemulsions and wash-off products such as soaps, shampoos, shower gels, face wash, conditioners. The excipients should be compatible and acceptable to the topical application.

The one or more pharmaceutically acceptable excipients may be selected from the group consisting of bases, oils, emulsifying agent or surfactant, cosurfactant, structurants, thickening agent, gelling agent, permeation enhancer, preservative, antioxidant, humectant, emollient, nourishing agents, neutralizers, rheology modifier, absorbent, opacifying agent, chelating agent, stabilizing agent, calming agent, sebum inhibitors, skin tightening agent, acidifying or alkalizing or buffering agent and vehicle.

The example of Bases includes but not limited to carnauba wax, cetyl alcohol, cetyl ester wax, emulsifying wax, hydrous lanolin, lanolin, lanolin alcohols, vegetable oils and animal fat; coconut oil, bees wax, olive oil, spermaceti wax, sesame oil, almond oil, alcohols, acids and esters; oleic acid, oleyl alcohol, palmitic acid, lauryl alcohol, lauric acid, myristyl alcohol, ethyl oleate, isopropyl myristicate, ethylene glycol, hydrogenated and sulphated oils; hydrogenated castor, cotton seed, hydrogenated sulphated castor oils, microcrystalline wax, liquid paraffin, petrolatum, polyethylene glycol, stearic acid, stearyl alcohol, white wax and yellow wax.

The examples of Oils according to present invention include but not limited to oleic acid, ethyl oleate, castor oil, corn oil, coconut oil, evening primrose oil, linseed oil, mineral oil, olive oil, peanut oil, clove oil, propylene dicaprylate/dicaprate glycol, glyceryl tricaprylate, isopropyl myristate and triglycerides (LCT, MCT, SCT) or combinations thereof.

The examples of Emulsifying agent or Surfactant according to present invention include but not limited to surfactants like spans and tweens, labrasol, labrafil, cremophor, poloxamer, emulsifying wax, glyceryl stearate, polyethylene glycol, PEG 100 stearate, sorbitan monostearate, sorbitan monooleate, sodium lauryl sulfate, propylene glycol monostearate, natural gums like acacia and tragacanth, hydrophilic colloids such as acacia and finely divided solids, e.g., bentonite and veegum, monovalent and bivalent soaps, lanolin, cholesterol or cholesterol esters, triethanolamine and its salts, dodecyl benzene sulfonate, diethylene glycol monoethyl ether and docusate sodium or combinations thereof.

The examples of Co-surfactant according to present invention include but not limited to transcutol, ethylene glycol, propylene glycol, ethanol, isopropanol, propylene glycol, glycerin, and PEG 400, dimethyl sulphoxide (DMSO) and dimethyl acetamide (DMA) or combinations thereof.

The examples of Structurants according to present invention include but not limited to fatty acids, fatty alcohols, stearic acid, fatty acid esters, and fatty acid amides, having fatty chains of from 8 to 30 carbons atoms.

The examples of Thickening agent include but not limited to carbomer, hydrogenated castor oil, methyl cellulose, sodium carboxyl methyl cellulose, carrageenan, colloidal silicon dioxide, natural gum such as gelatin, tragacanth gum and guar gum, hydroxypropyl cellulose, hydroxypropyl methyl cellulose, polyethylene oxide, alginic acid, paraffin, cetostearyl alcohol, PEG 200, PEG 300, PEG 400, PEG 600, monoethanolamine, triethanolamine, glycerol, propylene glycol, polyoxyethylene sorbiton monoleate, and poloxamers, polyvinyl pyrrolidone, various alcohols such as polyvinyl alcohol, ethanol or isopropyl alcohol, fumed silica.

The examples of Gelling agent includes but not limited to carbomer/carbopols, Pemulen®, cellulose derivatives such as methyl cellulose, hydroxy ethylcellulose, hydroxy propylmethylcellulose, carboxy methyl cellulose, hydroxy propyl cellulose, etc., glycerol, or propylene glycol gelled with suitable agents such as natural gums such as xanthan gum and tragacanth, fenugreek mucilage, pectin, poloxamers (pluronics), alginate, gelatin, starch, polyvinyl alcohol, povidone etc.

The example of Permeation enhancer includes but not limited to propylene glycol, ethanol, isopropyl alcohol, oleic acid, polyethylene glycol, phospholipids, cyclodextrins, black pepper (Piper nigrum), pyrrolidones, dimethyl sulphoxide (DMSO), decylmethyl sulphoxide (DCMS), terpenes (cineole, eugenol, D-limonene, menthol, menthone) and urea.

The examples of Preservatives include but not limited to chorocresol, benzoic acid, phenyl mercuric nitrate, benzyl alcohol, benzoic acid and its salts, boric acid, methyl paraben, propyl paraben, phenoxyethanol, trihydrate and anhydrous sodium acetate, chlorhexidine, formaldehyde, glutaraldehyde, imidazolidinyl urea, trichlosan, benzalkonium chloride and chloroxylenol.

The examples of Antioxidant includes but not limited to butylated hydroxyl anisole, butylated hydroxyl toluene, propyl gallate, polyols like sorbitol, xylitol and maltitol, natural extracts like Quillaia, or lactic acid or urea, lithium chloride, ascorbic acid, sodium metabisulfite, carotinoids, carotenes such as α-carotone, β-carotene and lycopene, chlorogenic acid, tocopherols, uric acid, ZnO and ZnSO₄.

The examples of Humectant includes but not limited to glycerin, glyceryl triacetate, propylene glycol, butylene glycol, sodium salt of pyroglutamic acid, polyethylene glycol, honey, hyaluronic acid, lecithin, sorbitol solution, sucrose, glucose and other sugars and their derivatives, lanolin, 1,2,6 hexanetriol, isopropyl myristate, petrolatum, isopropyl palmitate, hydrogenated castor oil, mineral oil, polyoxymethylene urea and potassium sorbate.

The examples of Emollient includes but not limited to cetyl palmitate, isopropyl myristate serate, esters, waxes, oils, castor oil, mineral oil, octyl dodecanol, cetyl alcohol, hexyl decanol, cetostearyl alcohol, cocoa butter, isopropyl myristate, isopropyl palmitate, lipids petrolatum, lanolin, liquid paraffin, polyethylene glycols, butters shea butter, silicone oils, stearic acid, and stearyl alcohol.

The examples of Nourishing agents includes but not limited to hyaluronic acid, coconut oil, collagen, biotin, protein, sweet almond oil, avocado oil, babassu oil, brazil nut oil, broccoli seed oil, buriti seed oil, castor oil, coco-caprylate/caprate, macadamia nut oil, mango seed butter, maracuja oil, moringa, nangai oil, rice bran oil, sunflower seed oil, tamanu oil, vitamin E oil and combination thereof. Hyaluronic acids (HA) for skin are polysaccharides (carbohydrates) that function to nourish collagen and elastin in the body. It fills out the free spaces between the collagen and elastin fibers to keep collagen flexible (on its own collagen doesn't have much stretch). HA acts as a nourishing agent to keep collagen limber and pliable. Hyaluronic acid helps skin cells survive longer, thickening the epidermis and preventing the build-up of dead skin in that layer. It may also protect the epidermis by scavenging free radicals generated by ultraviolet (UV) radiation exposure. Hyaluronic acid enhances moisture content of the skin. It also revitalizes skin's outer surface layers, so it looks and feels softer, smoother and radiantly hydrated. This instantly improves the appearance of fine lines and wrinkles. HA prevent a dry scalp and hair from thinning and eventually falling out. It is similar to a fertilizer because it can increase hair growth and the strand diameter-thicker hair.

Collagen is a fibrous, supportive protein. It is found in bone, cartilage, tendons, ligaments, and skin. Collagen production decreases with age, contributing to skin wrinkling and sagging. It helps skin cells adhere to one another and also gives the skin strength and elasticity. It acts as a film forming, moisturizing, nourishing, restructuring and protective agent. It makes possible a marked improvement of cutaneous hydration, elasticity and turgor. Due to the regular arrangement of fibrils, forming a thin macromolecular film on the horny layer, it is able to limit the unavoidable moisture losses caused by perspiration insensibilis, helping to maintain a full, fresh and smooth skin. Collagen is rich in amino acids that body needs to build keratin, the protein that makes up hair. Collagen makes up 70% of dermis, the middle layer of skin that contains the root of each individual hair. It contributes to the elasticity and strengthens dermis. Therefore, it may help maintain a healthy dermis and prevent hair thinning.

The examples of Neutralizers include but not limited to organic and inorganic neutralizers. Non-limiting examples of organic neutralizers are 2-amino-2-methyl-1, 3-propanediol (AMPD); 2-amino-2-ethyl-1, 3-propanediol (AEPD); 2-amino-2-methyl-1-propanol (AMP); 2-amino-1-butanol (AB); monoethanolamine (MEA); diethanolamine (DEA); triethanolamine (TEA); monoisopropanolamine (MIP A); diisopropanol-amine (DIP A); triisopropanolamine (TIP A); and dimethyl stearamine (DMS). A long chain amine neutralising agent such as stearamidopropyl dimethylamine or lauramidopropyl dimethyl amine may be employed. Also suitable are inorganic neutralisers, non-limiting examples of which include sodium hydroxide, potassium hydroxide and borax.

The examples of Rheology modifier include but not limited to Hydroxyethyl Acrylate/Sodium Acryloyldimethyl Taurate Copolymer (and) Isohexadecane (and) Polysorbate 60.

The examples of Absorbent include but not limited to magnesium carbonate, calcium carbonate, starch, cellulose and its derivatives.

The examples of Opacifying agent include but not limited to higher fatty alcohols such as cetyl, stearyl, cetostearyl alcohol, arachidyl and behenyl alcohols, solid esters such as cetyl palmitate, glyceryl laurate, various fatty acid derivatives such as propylene glycol and polyethylene glycol esters, inorganic materials such as, magnesium aluminum silicate, zinc oxide, titanium dioxide or other sunblocking agents.

The examples of Chelating agent include but not limited to ethylene diamine tetraacetate, dimercaprol, dimercaptosuccinic acid, penicillamine, deferoxamine, deferasirox, citric acid, maleic acid, phosphoric acid and like.

The examples of Stabilizing agent includes but not limited to max thick, waxes like candelila wax, cetyl alcohol, arrow root powder, behenyl alcohol, glucose oxidase, sclerotium gum, vegetable glycerides citrate, vegetable glycerides phosphate, microcellulose crystalline, diethylaminomethylcoumarin, disodium cupric citrate, disodium sebacate, ethyl dimethyl PABA, para-hydroquinone, hydroxycetyl phosphate, iron hydroxide, magnesium phosphate, methyl benzophenone and phenacetin or combination thereof.

The examples of Calming agent includes but not limited to menthyl lactate, allantoin, bisabolol, boerhavia diffusa root extract, burdock root, propionic acid derivatives, acetic acid derivatives, fenamic acid derivatives, colloidal oatmeal, curcumin and its derivatives, licorice and its components, especially glabridin, willow herb, green tea and its most active component, epigallocatechin gallate and lavender essential oil or combination thereof.

The examples of Sebum inhibitors include but not limited to aluminum hydroxy chloride, corticosteroids, dehydroacetic acid and its salts, dichlorophenyl imidazoldioxolan (available from Elubiol), mixtures thereof, and the like.

The examples of Skin tightening agent include but not limited to terpolymers of vinylpyrrolidone, (meth) acrylic acid and a hydrophobic monomer comprised of long chain alkyl (meth)acrylates, mixtures thereof, and the like.

The examples of Acidifying or Alkalizing or buffering agent includes but not limited to anhydrous and monohydrate citric acid, phosphoric acid, sodium hydroxide, potassium hydroxide, sodium bicarbonate, potassium sorbate, monobasic and dibasic sodium phosphate and trolamine.

The examples of Vehicle includes but not limited to purified water, hexylene glycol, propylene glycol, oleyl alcohol, propylene carbonate, mineral oil, almond oil, cottonseed oil, ethyl oleate, isopropyl myristate, isopropyl palmitate, myristyl alcohol, octyldodecanol, olive oil, peanut oil, safflower oil, sesame oil, soybean oil and squalene.

The anti-ageing composition comprising bioactive protein of the present invention can be manufactured by any suitable method known in the art such as high energy emulsification method: high pressure homogenizer, ultrasonication and microfluidization; low energy emulsification method: solvent evaporation technique, hydrogel method, phase inversion emulsification method, spontaneous emulsification; brute force method, simple solution, trituration method, levigation method, fusion method, chemical reaction method, emulsification method, thermal change method, flocculation or by simply incorporation of an emulsion into the gel base.

The concentration or amount of Bioactive protein and one or more pharmaceutically acceptable excipients, also the manufacturing process has been optimized in such way that topical bioactive protein containing composition provides reduction in the wrinkles on the skin, reduced sagging of skin, increased skin elasticity, reduction in dark spot formation, reduction of uneven skin tone, reduced fine line formation, skin firming and toning, moisturization and hydration, prevention of solar elastosis, reduction of skin tanning and darkening, reduced skin atrophy, increased skin repair, replenishment and rejuvenation achieved by extracellular matrix protein (such as collagen) induction, vascularization, melanin inhibition, better penetration, soothing effect, moisturizing effect, strengthening effect, calming effect, ease of application and spreadability, more efficacy and better patient compliance for the treatment of anti-ageing. Also provides reduction in hair loss, thinning of hair, dullness and loss of shine, loss of volume, loss of strength (brittleness), hair drying, increased number of grey hairs, hair shaft abnormalities, slow growth, decreased hair follicle and hair size.

The anti-ageing composition comprising bioactive protein, produced by XY- or XX-placenta explants, of according to present invention may be evaluated for efficacy evaluation like skin elasticity and firmness, anti-sagging, collagen fiber content, pigment content, sebum content, moisture content, under eye bags/puffiness, hair fall by comb's test, hair growth, smoothening and softness effect, sheen effect, split ends, strengthening effect and hair density.

### EXAMPLES

### Example 1

Skin cream for men comprising composition of bioactive protein XY-placenta. Ingredients (weight %):

Glycerin (humectant): 5.0
Disodium EDTA (chelating agent): 0.10
Cetyl palmitate (moisturizer): 5.0
Glyceryl stearate (emulsifier): 5.0
PEG 100 stearate (co-emulsifier): 2.0
Isopropyl myristate serrate (emollient): 1.0
Medium chain coconut oil (nourishment): 1.0
Polysorbate (solubilizer): 0.5
Phenoxyethanol (preservative): 2.0
Hyaluronic acid (nourishment): 2.0
Collagen (restorative nourishment): 1.0
Biotin (restorative nourishment): 3.0
Biological active proteins (anti-ageing agents in conditional medium): 7.2
Max thick (stabilizer): 5.0
Methyl lactate (calming agent): 0.5
Water QS (base): 59.7
Biological active proteins (final concentrations in cream):
EGF, pg/1 ml of cream: 1.4
FGF-7, pg/1 ml of cream: 23.3
HGF, pg/1 ml of cream: 158.1
IGF-1, ng/1 ml of cream: 72.1
VEGF-A, ng/1 ml of cream: 3.0
SCF, pg/1 ml of cream: 1.9
TGF-beta 1, ng/1 ml of cream: 13.8
TNF-alpha, pg/1 ml of cream: 40.4
ANGPT1, pg/1 ml of cream: 13.2
bFGF, pg/1 ml of cream: 7.5

The stability of biologically active proteins of the XY-placenta and their compatibility with the cosmetic components of a male skin cream was studied under three experimental conditions: when the cream samples were stored for 12 months at room temperature (24±2° C.), in a freezer (4±0.2° C.), and in a thermostat (37±0.1° C.). Biologically active protein concentrations were determined by ELISA after extraction with acetonitrile/ethyl acetate. The control was the content of biologically active peptides in 1 ml of freshly prepared male skin cream (100%). The concentration of biologically active proteins after storage was (FIG. 6): when stored at room temperature—97±1.5% (94-100%), when stored in a fridge—98±1% (96-100%), when stored in a thermostat—93±3% (87-99%). This indicates a sufficient stability and compatibility of biologically active proteins with the cosmetic components of the male skin cream.

Safety and efficacy of skin cream for men comprising composition of bioactive protein XY-placenta have been validated in pilot clinical trials. This was an observational study wherein 20 otherwise healthy male volunteers were selected, with the chief complaint of dull and aging skin. The total duration of the study was 90 days. Volunteers were given the cream to be applied on the face once every night. In the dynamics of observation, such indicators as skin elasticity and firmness, anti-sagging, collagen fiber content, pigment content, sebum content, moisture content, under eye bags/puffiness, Global Assessment Scale by the investigator and the volunteers were studied (Ennen et al., 2014; Escoffier C et al., 1989; Varani J et al., 2001; Shuster S, 1975; Langley R G B et al., 2013).

Safety evaluation. No serious side effects were observed within 3 months of observation. None of the volunteers reported signs of skin irritation, allergic reactions, or any other side effects.

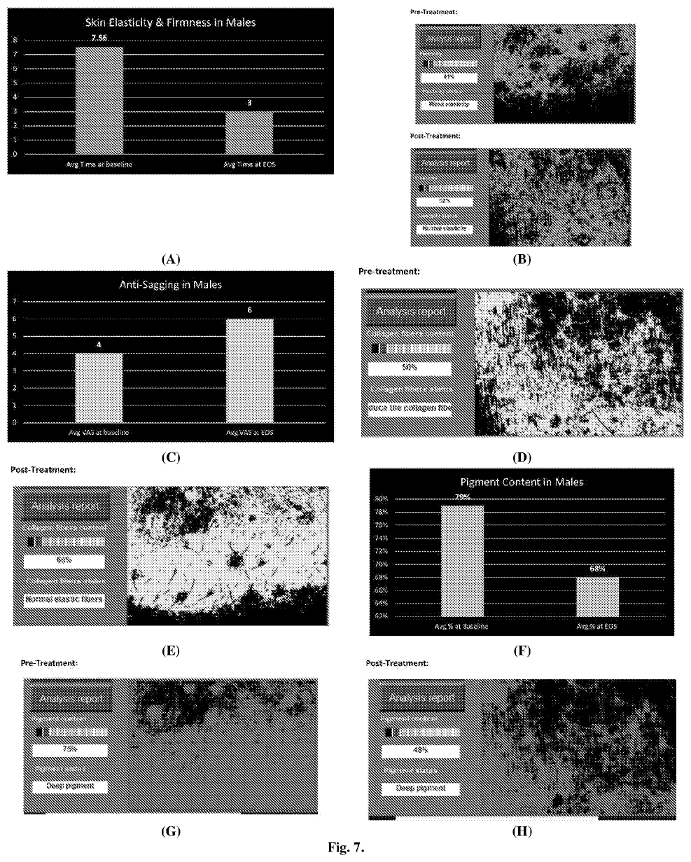

The skin elasticity and firmness were assessed by a pinch test from the baseline to the end of the study. In the initial period, the mean time of the pinch test was 7.56±0.75 seconds and significantly decreased at the end of the study (FIG. 7A)—to 3.41±0.38 seconds (p<0.001; n=20).

It was also observed that the average skin elasticity was 41.2±3.15% in pre-application period which was interpreted as skin moderately damaged due to sun, pollution or other external and ageing factors. It was showed improved of skin elasticity to 52.9±3.06% (p<0.02; n=20) in post cream application time which was interpreted as good skin glow and sheen (FIG. 7B).

In case of anti-sagging the treating doctor's feedback was taken on a VAS scale (where 0 was worst sagging, 1-4 was severe sagging, 5 was moderate sagging, 6-9 was minimum sagging and 10 was no sagging) based on the visual examination during the time of the visit. It was observed that the average VAS score before the start of the study was 4.11±0.33. Post the application the VAS scores improved and were increased to 6.98±0.72 (p<0.001; n=20) where there was only minimum sagging observed by the treating doctor (FIG. 7C).

The collagen fiber content showed an increase from 50.2±3.63% to 66.8±4.98% (p<0.02; n=20) on an average during the course of the study (FIG. 7D, E).

There was marked reduction in the pigment content of the skin (study population—Indian people) from 79.3±3.41% on an average prior to cream application to 68.1±2.75% (p<0.02; n=20) at the end of the study (FIG. 7F). For example, in volunteer C the pigment content showed a reduction of 27%, from 75% pre-treatment to 48% post-treatment (FIG. 7G, H).

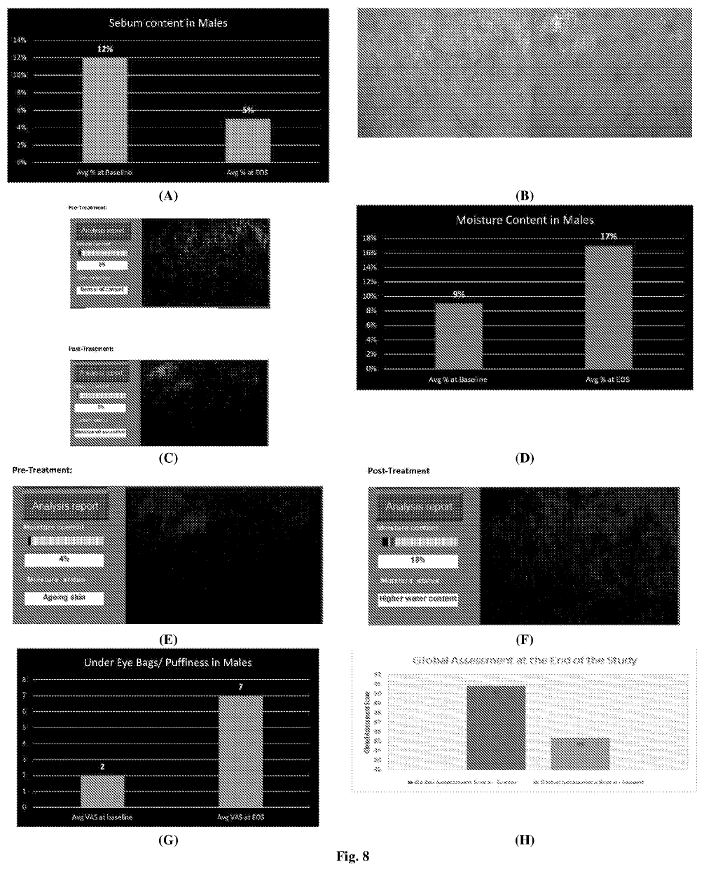

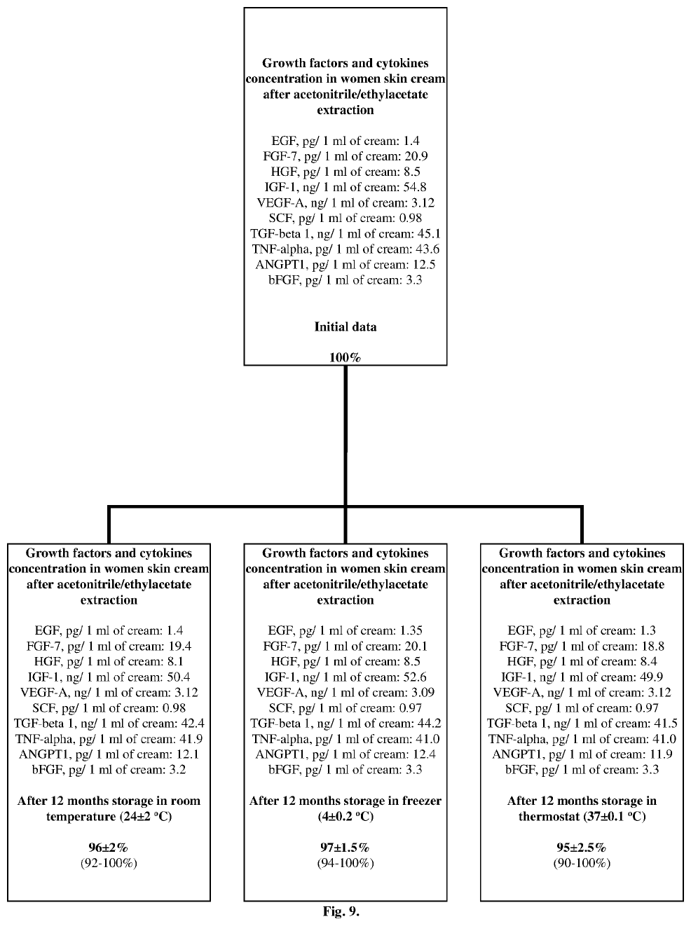

After using the cream, men showed a significant decrease in sebum content: from 12.20±1.98% at the beginning of the study to 5.65±0.63% (p<0.01; n=20) at the end of the study (FIG. 8A, B). In cases with moderate sebum content, this indicator decreased from 6 to 3%, that is, the sebum content decreased twice (FIG. 8C).

The cream showed a good moisturizing effect (FIG. 8D): the moisture content in the skin increased from the initial level of 9.3±0.88% to 17.5±1.12% at the end of the study (p<0.001; n=20). In some cases, the moisture content of the skin increased more when 4 times, rising from a baseline of 4% to 18% at the end of the study (FIG. 8E, F).

In case of under eye puffiness the treating doctor's feedback was taken on a VAS scale (where 0 was heavy puffiness, 1-4 was severe puffiness, 5 was moderate puffiness, 6-9 was minimum puffiness and 10 was no puffiness) based on the visual examination during the time of the visit. It was observed (FIG. 8G) that the average VAS score before the start of the cream application was 2.13±0.24. Post the application of cream the VAS scores indicators improved and were increased to 7.46±0.79 (p<0.001; n=20).

At the end of the study, global assessment was done by the investigator and the volunteers. The following graph represents the Global Assessment Score (FIG. 8H), which indicates high efficiency of skin cream for men comprising composition of bioactive protein XY-placenta.

### Example 2

Skin cream for women comprising composition of bioactive protein XX-placenta. Ingredients (weight %):

Glycerin (humectant): 5.0
Disodium EDTA (chelating agent): 0.10
Cetyl palmitate (moisturizer): 5.0
Glyceryl stearate (emulsifier): 5.0
PEG 100 stearate (co-emulsifier): 2.0
Isopropyl myristate serrate (emollient): 1.0
Medium chain coconut oil (nourishment): 1.0
Polysorbate (solubilizer): 0.5
Phenoxyethanol (preservative): 2.0
Hyaluronic acid (nourishment): 2.0
Collagen (restorative nourishment): 1.0
Biotin (restorative nourishment): 3.0
Biological active proteins (anti-ageing agents in conditional medium): 7.2
Max thick (stabilizer): 5.0
Methyl lactate (calming agent): 0.5
Water QS (base): 59.7
Biological active proteins (concentrations):
EGF, pg/1 ml of cream: 1.5
FGF-7, pg/1 ml of cream: 22.0
HGF, pg/1 ml of cream: 8.7
IGF-1, ng/1 ml of cream: 57.0
VEGF-A, ng/1 ml of cream: 3.25
SCF, pg/1 ml of cream: 1.0
TGF-beta 1, ng/1 ml of cream: 46.0
TNF-alpha, pg/1 ml of cream: 45.5
ANGPT1, pg/1 ml of cream: 13.0
bFGF, pg/1 ml of cream: 3.6

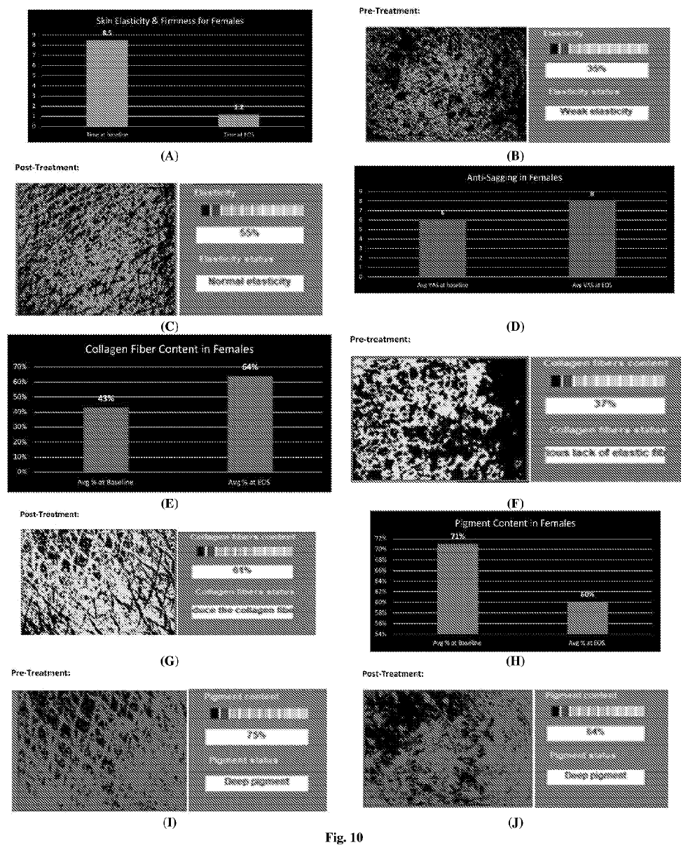

The stability of biologically active proteins of XX-placenta and their compatibility with the cosmetic components of the female skin cream was studied under three experimental conditions: when the cream samples were stored for 12 months at room temperature (24±2° C.), in a freezer (4±0.2° C.), and in a thermostat (37±0.1° C.). Biologically active protein concentrations were determined by ELISA after extraction with acetonitrile/ethyl acetate. The control was the content of biologically active peptides in 1 ml of freshly prepared female skin cream (100%). The concentration of biologically active proteins after storage was (FIG. 9): when stored at room temperature—96±2% (92-100%), when stored in a fridge—97±1.5% (94-100%), when stored in a thermostat—95±2.5% (90-100%). This indicates a sufficient stability and compatibility of biologically active proteins with the cosmetic components of the female skin cream.

Safety and efficacy of skin cream for women comprising composition of bioactive protein XX-placenta have been validated in pilot clinical trials. This was an observational study wherein 20 otherwise healthy female volunteers were selected, with the chief complaint of dull and aging skin. The total duration of the study was 90 days. Volunteers were given the cream to be applied on the face once every night. In the dynamics of observation, such indicators as skin elasticity and firmness, anti-sagging, collagen fiber content, pigment content, sebum content, moisture content, under eye bags/puffiness, Global Assessment Scale by the investigator and the volunteers were studied (Ennen et al., 2014; Escoffier C et al., 1989; Varani J et al., 2001; Shuster S, 1975; Langley R G B et al., 2013).

Safety evaluation. No serious side effects were observed within 3 months of observation. None of the volunteers reported signs of skin irritation, allergic reactions, or any other side effects.

The elasticity and firmness of the skin was assessed by the pinch test (FIG. 10A). At the end of the study, the mean time during which the skin flattens after 5 seconds of pinching the skin was sevenfold lower than baseline: 8.54±0.76 seconds at the start of the study versus 1.21±0.09 seconds (p<0.001; n=20).

It was also observed that the average skin elasticity was 31.23±2.61% pre-application of cream which was interpreted as skin moderately damaged due to sun, pollution or other external factors. It was observed that the average was improved to 40.95±3.08% post cream application (p<0.05; n=20) which was interpreted good skin glow and sheen. The skin showed weak elasticity at the start of the study, while normal elasticity was achieved by the end of the study. In some women the skin showed 20% improvement from the baseline to the end of the study, from 35% to 55% (FIG. 10B, C).

In case of anti-sagging the treating doctor's feedback was taken on a VAS scale (where 0 was worst sagging, 1-4 was severe sagging, 5 was moderate sagging, 6-9 was minimum sagging and 10 was no sagging) based on the visual examination during the time of the visit. It was observed that the average VAS score before the start of the study was 6.18±0.35. Post the application the VAS scores improved and were increased to 8.96±0.46 (p<0.001; n=20) where there was only minimum sagging observed by the treating doctor (FIG. 10D).

The collagen fiber content showed an increase from 43.08±2.51% to 64.97±3.12% (p<0.001; n=20) on an average during the course of the study (FIG. 10E). For example, in volunteer A pre-treatment the skin showed a lack of collagen fibers, which increased significantly post-treatment: the collagen fiber content pre-treatment was 37% while it was 61% post-treatment (FIG. 10F, G).

There was marked reduction in the pigment content of the skin (study population—Indian people) from 71.85±2.95% on an average prior to cream application to 60.46±1.94% (p<0.01; n=20) at the end of the study (FIG. 10H). For example, in volunteer B the pigment content showed a reduction of 9%, from 75% pre-treatment to 64% post-treatment (FIG. 10I, J).

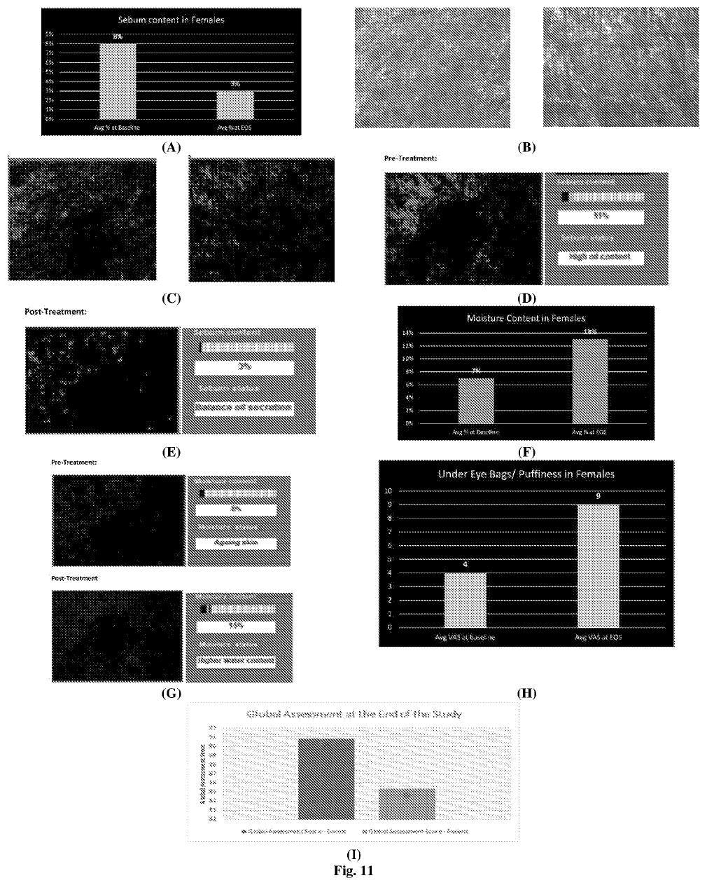

After using the cream, women showed a significant decrease in sebum content: from 8.31±0.59% at the beginning of the study to 3.47±0.51% (p<0.001; n=20) at the end of the study (FIG. 11A). For some volunteers with a high sebum content, this indicator decreased from 10 to 6%, that is, the sebum content decreased by 4% (FIG. 11B, C). In others cases, the decrease in sebum content was more significant: from 11% to 3%, that is, after using the cream, the sebum content decreased by 8% (FIG. 11D, E).

The cream showed a good moisturizing effect (FIG. 11F): the moisture content in the skin increased from the initial level of 7.42±0.69% to 13.75±1.86% at the end of the study (p<0.01; n=20). In some cases, the moisture content of the skin increased almost twice, rising from a baseline of 8% to 15% at the end of the study (FIG. 11G).

In case of under eye puffiness the treating doctor's feedback was taken on a VAS scale (where 0 was heavy puffiness, 1-4 was severe puffiness, 5 was moderate puffiness, 6-9 was minimum puffiness and 10 was no puffiness) based on the visual examination during the time of the visit. It was observed (FIG. 11H) that the average VAS score before the start of the cream application was 4.27±0.51. Post the application of cream the VAS scores indicators improved and were increased to 9.62±0.98 (p<0.001; n=20).

At the end of the study, global assessment was done by the investigator and the volunteers. The following graph represents the Global Assessment Score (FIG. 11I), which indicates high efficiency of skin cream for women comprising composition of bioactive protein XX-placenta.

### Example 3

Hair lotion for men comprising composition of bioactive protein XY-placenta. Ingredients (weight %):

Glycerin (humectant): 0.1
Disodium EDTA (chelating agent): 0.01
Cetyl palmitate (moisturizer): 0.1
Glyceryl stearate (emulsifier): 0.1
PEG 100 stearate (co-emulsifier): 0.1
Isopropyl myristate serrate (emollient): 0.1
Medium chain coconut oil (nourishment): 0.1
Polysorbate (solubilizer): 0.1
Phenoxyethanol (preservative): 0.1
Hyaluronic acid (nourishment): 0.1
Collagen (restorative nourishment): 0.1
Biotin (restorative nourishment): 0.1
Biological active proteins (anti-ageing agents in conditional medium): 7.2
Max thick (stabilizer): 1.0
Methyl lactate (calming agent): 0.1
Water QS (base): 90.59
Biological active proteins (concentrations):
EGF, pg/1 ml of lotion: 1.4
FGF-7, pg/1 ml of lotion: 23.8
HGF, pg/1 ml of lotion: 162.0
IGF-1, ng/1 ml of lotion: 74.0
VEGF-A, ng/1 ml of lotion: 3.0
SCF, pg/1 ml of lotion: 1.9
TGF-beta 1, ng/1 ml of lotion: 14.1
TNF-alpha, pg/1 ml of lotion: 41.3
ANGPT1, pg/1 ml of lotion: 13.5
bFGF, pg/1 ml of lotion: 7.7

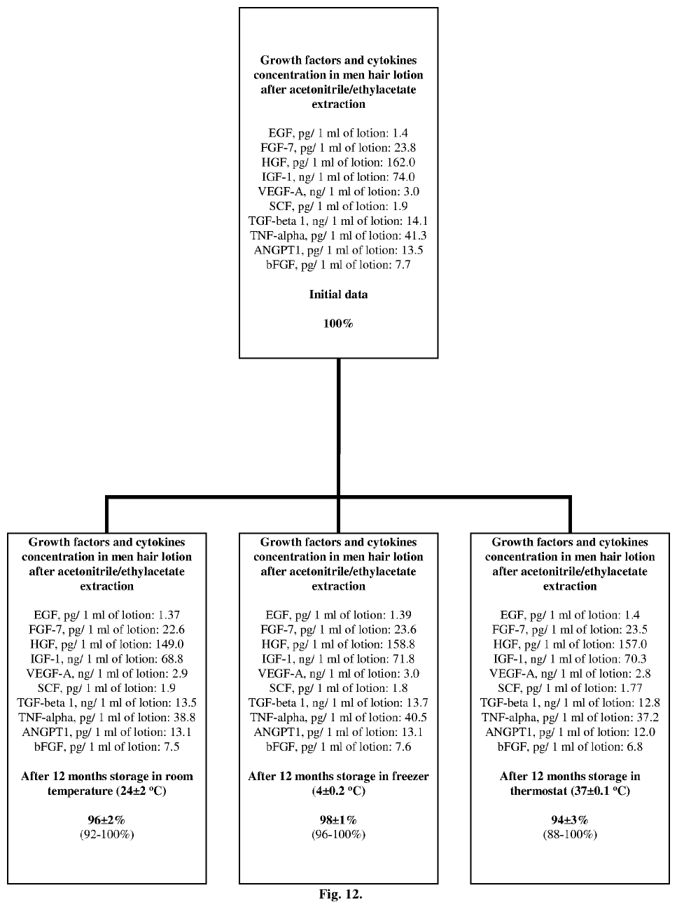

The stability of biologically active proteins of XY-placenta and their compatibility with the cosmetic components of the male hair lotion was studied under three experimental conditions: when the cream samples were stored for 12 months at room temperature (24±2° C.), in a freezer (4±0.2° C.), and in a thermostat (37±0.1° C.). Biologically active protein concentrations were determined by ELISA after extraction with acetonitrile/ethyl acetate. The control was the content of biologically active peptides in 1 ml of freshly prepared male hair lotion (100%). The concentration of biologically active proteins after storage was (FIG. 12): when stored at room temperature—96±2% (92-100%), when stored in a fridge—98±1% (96-100%), when stored in a thermostat—94±3% (88-100%). This indicates a sufficient stability and compatibility of biologically active proteins with the cosmetic components of the male hair lotion.

Safety and efficacy of hair lotion for men comprising composition of bioactive protein XY-placenta have been validated in pilot clinical trials. This was an observational study wherein 40 otherwise healthy male volunteers were selected, with the chief complaint of hair fall and dull hair. The total duration of the study was 60 days. Volunteers were given the hair lotion, that had to be applied on the scalp every alternate night for a duration of 2 months. A comb's test was performed to access the hair fall at the baseline and end of study. The hair analyzer was used to access the hair growth/sq·cm. The smoothening and softness of the hair were evaluated by a questionnaire survey by the volunteers, wherein the volunteers had to score the smoothening and softness of hair on a scale of 0-10, 10 being very smooth and soft hair. The hair sheen was also evaluated based on the scores of a questionnaire survey completed by the volunteers. The average score of 5 at the baseline improved to 6 at the end of the study (0—no sheen, 1-4—lacking sheen, 5—moderate sheen, 6-9—good sheen, 10—best sheen). The hair strength was assessed by the pull test, where the number of hairs plucked from the scalp in one pinch was calculated. The hair density as measured with the help of the hair (Dhurat R, Saraogi P., 2009).

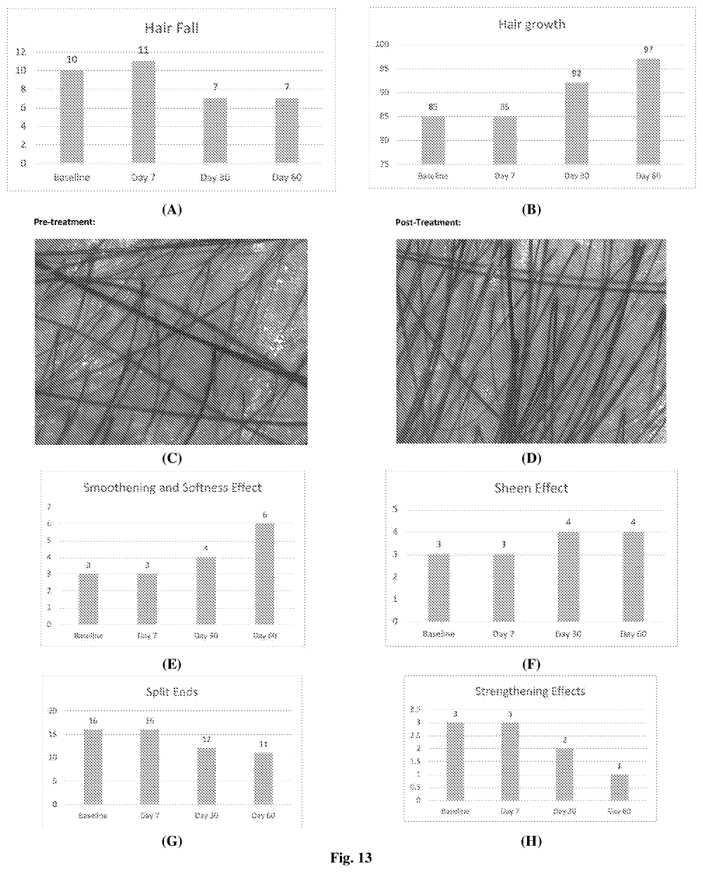

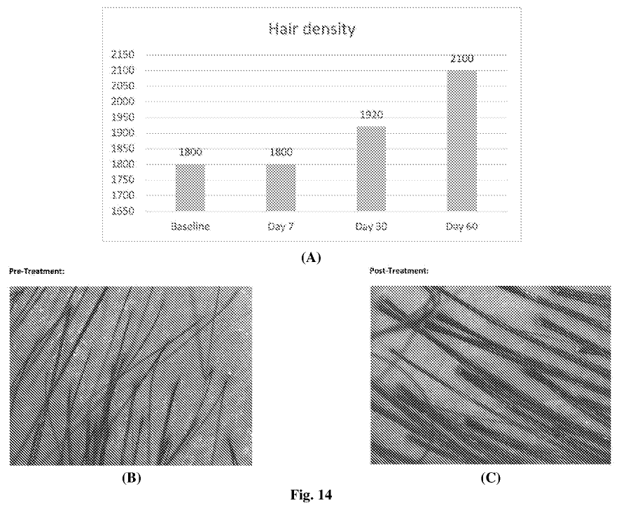

A comb's test was performed to access the hair fall at the baseline and end of study. It was observed (FIG. 13A) that the average hair falls of 10.80±1.02 hair/combing at the baseline visit was considerably reduced to 7.21±0.63 hair/combing by the end of the study (p<0.01; n=40).

It was observed that there was consistent improvement in the hair growth of the subjects along the study (FIG. 13B): at baseline the hair growth was 85.5±3.43 hair/sq·cm on an average which had increased to 97.9±5.17 hair/sq·cm on an average by the end of the investigation (p<0.05; n=40). The photo-image shows example of the increase in the number of new hair growth before and after the using hair lotion (FIG. 13C, D).

The smoothening and softness of the hair were evaluated by a questionnaire survey by the volunteers, wherein the volunteers had to score the smoothening and softness of hair on a scale of 0-10 (0—being very rough hair; 1-4—hair lacking smoothness and softness; 5—is moderately soft and smooth hair; 6-9—being smooth and soft hair; and 10—being very smooth and soft hair). Based on this feedback, it was observed (FIG. 13E) that the average score of 3.20±0.29 at the baseline visit had improved to 6.78±0.65 by the end of the study (p<0.001; n=40).

The hair sheen was also evaluated based on the scores of a questionnaire survey completed by the volunteers (FIG. 13F). The average score of 3.11±0.32 at the baseline improved to 4.89±0.46 at the end of the study (p<0.01; n=40).

Based on the assessment by the investigator (FIG. 13G), the average number of hairs with split ends in 1 cm² at the baseline was 16.79±1.48, which at the end of the study was reduced to 11.22±0.98 (p<0.01; n=40).

The hair strength was assessed by the pull test, where the number of hairs plucked from the scalp in one pinch was calculated (FIG. 13H). The average score of 3.78±0.39 at the baseline visit had reduced to 1.21±0.18 at the end of the study (p<0.001; n=40).

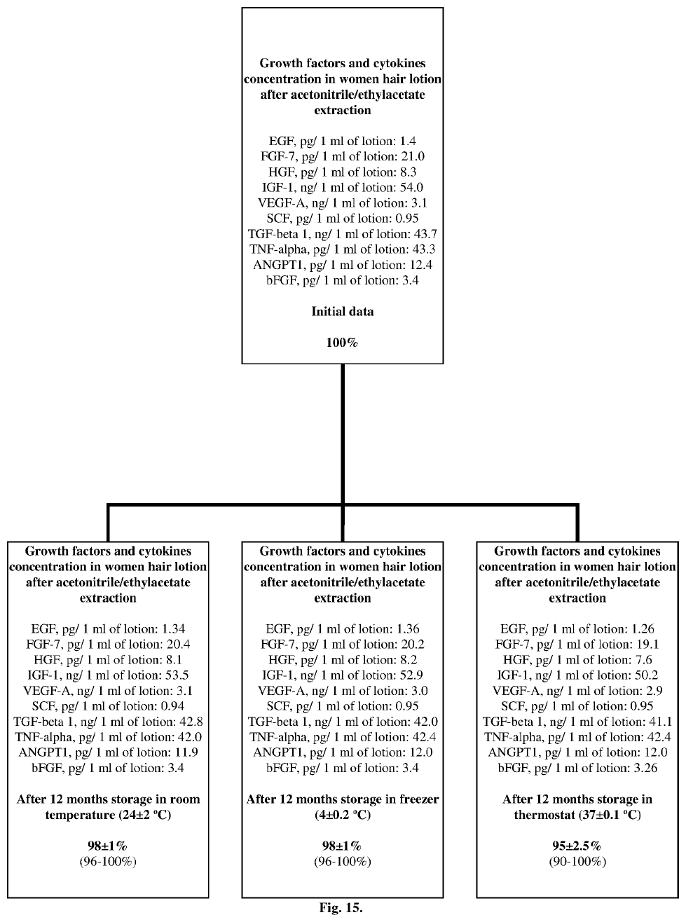

The hair density too showed an improvement during the duration of the study (FIG. 14A). At baseline the average hair density was 1800±78 hair/sq. inch while by the end of the study the average hair density had increased to 2100±95 hair/sq. inch (p<0.02; n=40). The increase in hair density can be appreciated in this example photo before and after the completion of the study (FIG. 14B, C).

Safety evaluation. There were no serios adverse event and side effects expected to be observed during the 2 months of observation period. During the entire observation period none of the participants report any side effect (skin irritation, dry scalp skin, allergic reaction, dandruff and others). Thus, pilot clinical trial showed the safety and high efficiency of hair lotion for men comprising composition of bioactive protein XY-placenta.

### Example 4

Hair lotion for women comprising composition of bioactive protein XX-placenta. Ingredients (weight %):

Glycerin (humectant): 0.1
Disodium EDTA (chelating agent): 0.01
Cetyl palmitate (moisturizer): 0.1
Glyceryl stearate (emulsifier): 0.1
PEG 100 stearate (co-emulsifier): 0.1
Isopropyl myristate serrate (emollient): 0.1
Medium chain coconut oil (nourishment): 0.1
Polysorbate (solubilizer): 0.1
Phenoxyethanol (preservative): 0.1
Hyaluronic acid (nourishment): 0.1
Collagen (restorative nourishment): 0.1
Biotin (restorative nourishment): 0.1
Biological active proteins (anti-ageing agents in conditional medium): 7.2
Max thick (stabilizer): 1.0
Methyl lactate (calming agent): 0.1
Water QS (base): 90.59
Biological active proteins (concentrations):
EGF, pg/1 ml of lotion: 1.4
FGF-7, pg/1 ml of lotion: 21.0
HGF, pg/1 ml of lotion: 8.3
IGF-1, ng/1 ml of lotion: 54.0
VEGF-A, ng/1 ml of lotion: 3.1
SCF, pg/1 ml of lotion: 0.95
TGF-beta 1, ng/1 ml of lotion: 43.7
TNF-alpha, pg/1 ml of lotion: 43.3
ANGPT1, pg/1 ml of lotion: 12.4
bFGF, pg/1 ml of lotion: 3.4

The stability of biologically active proteins of XX-placenta and their compatibility with the cosmetic components of female hair lotion was studied under three experimental conditions: when the cream samples were stored for 12 months at room temperature (24±2° C.), in a freezer (4±0.2° C.), and in a thermostat (37±0.1° C.). Biologically active protein concentrations were determined by ELISA after extraction with acetonitrile/ethyl acetate. The control was the content of biologically active peptides in 1 ml of freshly prepared female hair lotion (100%). The concentration of biologically active proteins after storage was (FIG. 15): when stored at room temperature—98±1% (96-100%), when stored in a fridge—98±1% (96-100%), when stored in a thermostat—95±2.5% (90-100%). This indicates a sufficient stability and compatibility of biologically active proteins with the cosmetic components of the female hair lotion.

Safety and efficacy of hair lotion for women comprising composition of bioactive protein XX placenta have been validated in pilot clinical trials. This was an observational study wherein 40 otherwise healthy female volunteers were selected, with the chief complaint of hair fall and dull hair. The total duration of the study was 60 days. Volunteers were given the hair lotion, that had to be applied on the scalp every alternate night for a duration of 2 months. A comb's test was performed to access the hair fall at the baseline and end of study. The hair analyzer was used to access the hair growth/sq·cm. The smoothening and softness of the hair were evaluated by a questionnaire survey by the volunteers, wherein the volunteers had to score the smoothening and softness of hair on a scale of 0-10, 10 being very smooth and soft hair. The hair sheen was also evaluated based on the scores of a questionnaire survey completed by the volunteers. The average score of 5 at the baseline improved to 6 at the end of the study (0—no sheen, 1-4 —lacking sheen, 5—moderate sheen, 6-9—good sheen, 10—best sheen). The hair strength was assessed by the pull test, where the number of hairs plucked from the scalp in one pinch was calculated. The hair density as measured with the help of the hair (Dhurat R, Saraogi P., 2009).

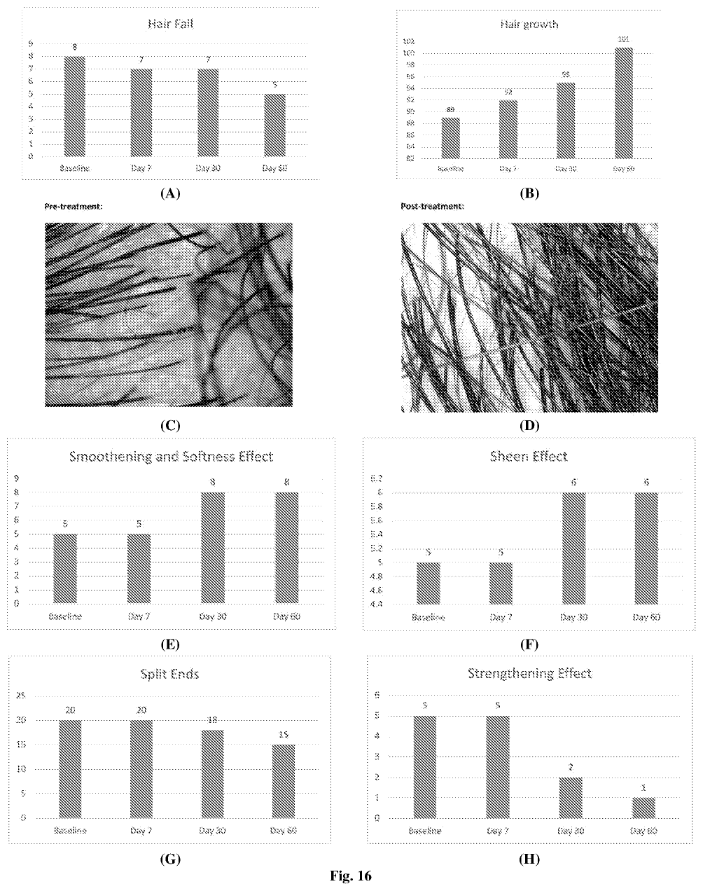

A comb's test was performed to access the hair fall at the baseline and end of study. It was observed (FIG. 16A) that the average hair falls of 8.47±0.78 hair/combing at the baseline visit was considerably reduced to 5.06±0.42 hair/combing at the end of the study (p<0.001; n=40).

It was observed (FIG. 16B) that there was consistent improvement in the hair growth of the female subjects, during each of the visits. At baseline the hair growth was 89.0±2.63 hair/sq·cm on an average which had increased to 101.9±5.40 hair/sq·cm on an average by the end of the study (p<0.05; n=40). As evident in the photo-images there was an increase in the number of new hair growth after completion of treatment (FIG. 16C, D).

The smoothening and softness of the hair were evaluated by a questionnaire survey by the volunteers, wherein the volunteers had to score the smoothening and softness of hair on a scale of 0-10 (0 being very rough hair; 1-4—hair lacking smoothness and softness; 5—is moderately soft and smooth hair; 6-9—being smooth and soft hair; and 10—being very smooth and soft hair). Based on this feedback, it was observed (FIG. 16E) that the average score of 5.10±0.54 at the baseline visit had improved to 8.98±0.96 by the end of the study (p<0.001; n=40).

The hair sheen was evaluated by the scores of a questionnaire survey completed by the volunteers (FIG. 16F). The average score of 5.21±0.53 at the baseline improved to 6.97±0.62 at the end of the study (p<0.05; n=40).

Based on the assessment by the investigator (FIG. 16G), the average number of hairs with split ends in 1 cm² at the baseline was 20.7±1.92, which at the end of the study was reduced to 15.2±1.54 (p<0.05; n=40).

The hair strength was assessed by the pull test, where the number of hairs plucked from the scalp in one pinch was calculated (FIG. 16H). The average score of 5.74±0.65 at the baseline visit had reduced to 1.60±0.25 at the end of the study (p<0.001; n=40).

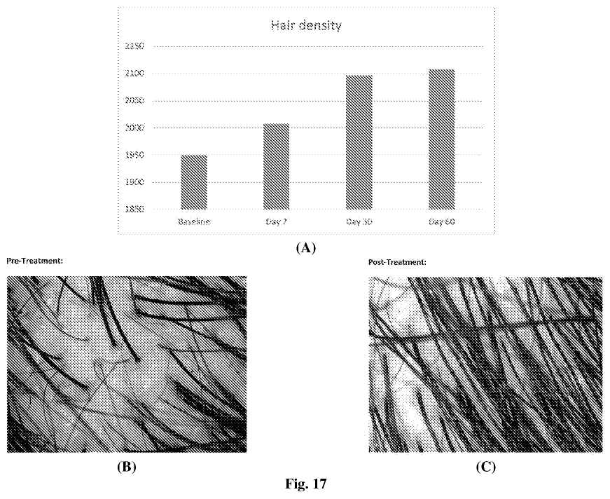

The hair density also showed an improvement along the study (FIG. 17A). At baseline the average hair density was 1950±45 hair/sq. inch while by the end of the study the average hair density had increased to 2108±58 hair/sq. inch (p<0.05; n=40). The increase in hair density can be appreciated in this example photo before and after the completion of the study (FIG. 17B, C).

Safety evaluation. There were no serios adverse event and side effects expected to be observed during the 3 months of observation period. During the entire observation period none of the participants report any side effect (skin irritation, dry scalp skin, allergic reaction, dandruff and others). Thus, pilot clinical trial showed the safety and high efficiency of hair lotion for women comprising composition of bioactive protein XX-placenta.

### Discussion

The present invention relates to the method of obtaining a composition of biologically active proteins from an abortive placenta at the term of medical termination of pregnancy 11-12 weeks. Obtaining biologically active proteins is achieved by cultivating the explants of the abortive XY-placenta or XX-placenta. The placenta plays a key role in fetal development and influences the programming of fetal development. Sex-specific regulatory pathways that control dimorphic characteristics in various organs and tissues are associated with the abundance of genes associated with the X-chromosome involved in placentogenesis, as well as with early uneven gene expression by sex chromosomes in men and women, and the role of genes linked to X- and Y-chromosomes, especially those involved in the expression of the placenta specific genes (Gabory A. et al, 2013). The placenta is also a major endocrine organ being responsible for synthesizing vast quantities of hormones and cytokines that have important effects on both maternal and fetal physiology (John R, Hemberger M., 2012; Roseboom T J et al., 2011; Lahti J et al., 2009; Thornburg K L et al., 2010).

Most of the studies focusing on the placenta have not taken the sex of the embryo into account. However, as trophoblast cells originate from the embryo, they reflect fetal sex as either XX or XY, allowing for possible sex differences in placental biochemistry, function, and signaling. Ishikawa et al. (2003) have clearly established an effect of sex chromosome “dosage”, independent of androgen, on placental size in mice, with XX-placentas being significantly smaller than XY-placentas. Although the presence of two X chromosome rather than one leads to a decrease in placental size, the basic mechanism is still to be clarified. Moreover, all males' cells possess a single X chromosome of maternal origin and a Y chromosome of paternal origin while female cells consist of two population, one with inactive maternally inherited X and the other with inactive paternally inherited X. Further, X chromosomes in the placenta could be reactivated or inactivated in response to intrauterine conditions (Migeon B R et al., 2005). This plasticity in X-inactivation in the placenta may be an important contributor to sex-differences in response to environmental perturbations during gestation, whereby females may be buffered from detrimental conditions to a greater degree than males due to increased expression of important X-linked genes. Many studies (Vatten L J, Skjaerven R., 2004; Di Renzo G C et al., 2007; Engel P J et al., 2008; Stevenson D K et al., 2000; Clifton V L, Vanderlelie J, Perkins A V., 2005) have observed gender specific differences in fetal growth and fetal and neonatal morbidity and mortality.

The sex of the embryo affects the size of both the fetus and the placenta, together with the ability of the placenta to respond to adverse stimuli (Mao J et al., 2010). The placenta has traditionally been considered an asexual organ and therefore, many studies focusing on the placenta have not taken the sex of the embryo into account. But given its extraembryonic origin, the placenta has a sex: that of the embryo it belongs to (Clifton V L, 2005, 2010) and numerous DOHaD studies indicate that sex differences can originate early in development and in particular in the placenta (Clifton V et al., 2009). Studies have clearly established an effect of sex chromosome “dosage” on placental size in mice, with XY placentas being significantly larger than XX placentas and that such differences are independent of androgen effects (Moritz K M et al., 2010).

Gender differences were observed in the placenta at multiple levels: epigenetic modifications of DNA, gene expression, protein expression and immune function. Epigenetic modifications of DNA occur without any alteration in the underlying DNA sequence and can control whether a gene is turned on or off and how much of a specific message is transmitted. Every cell in the body has the same DNA sequence but different genes are turned on or off to make different tissues, such as skin, kidney or liver. The placenta is influenced by numerous environmental factors including nutrients and tissue oxygenation, which may modify epigenetic marks and gene expression within the placenta and consequently placental development and function. The resulting alterations in epigenetic marks may alter cell fate decisions, the subsequent growth and development of tissues and organs. The sex of the placenta and the environment affects its epigenomes, and therefore the epigenomes of the developing fetus. In many adult tissues, such as gonads, brain, and liver, the expression of numerous genes is regulated in a sex-specific manner (Gabory A et al., 2013; Qureshi I A, Mehler M F., 2010; Waxman D J, Holloway M G., 2009). At present, the three major epigenetic factors are post-translational histone modifications, DNA methylation, and small noncoding RNAs, such as microRNAs (miRNAs). All of these processes are closely connected. For example, changes in histone marks, such as acetylation or methylation, are necessary for DNA methylation and demethylation to occur (Cedar H, Bergman Y., 2009). DNA methylation can regulate miRNA expression and vice versa [Han L et al., 2007; Wu L et al., 2010], and miRNA frequently target and regulate levels of the histone-modifying enzymes deacetylases and methyltransferases (Guil S, Esteller M., 2009; Tuddenham L et al., 2006; Varambally S et al., 2008; Wong C F, Tellam R L., 2008). The exact location and combination of these modifications determines small- and large-scale chromatin conformational changes (Jenuwein T, Allis C D., 2001).

MiRNAs are highly conserved, regulatory molecules that have an important role in the post-transcriptional regulation of target gene expression by promoting mRNA instability or translational inhibition [Inui M et al., 2010]. MiRNAs are expressed in placenta and alterations in their expression have been described in association with exposure to xenobiotics (Avissar-Whiting M et al., 2010), cigarette smoking (Maccani M A et al., 2010) or with adverse pregnancy outcomes including preeclampsia (Enquobahrie D A et al., 2011) and growth restriction (Maccani M A, Padbury J F, Marsit C J., 2011). Moreover, they may play a role in regulating sex specific gene expression. One seminal study, that set the de novo landscape of miRNA-regulation in cells of the trophoblast lineage, was published in 2012 by Morales-Prieto et al. In this study, the authors screened 762 human miRNAs for their expression level in term and first trimester cytotrophoblasts. One of the major outcomes of this work was the identification of clusters of placenta-specific miRNAs (C19MC, 54 miRNAs on chromosome 19, C14MC, 34 miRNAs on chromosome 14, and another minor cluster on chromosome 19). Placenta-specific miRNAs epigenetically regulate the expression of gene sets associated with both adaptive and innate immune responses throughout pregnancy, while miRNAs controlling oncogenic, angiogenic and anti-apoptotic genes appear dominant during the first trimester, miRNAs promoting cell differentiation are highly expressed in late pregnancy (Gu Y et al., 2013).

In mice and cattle, accelerated development is already evident in XY blastocysts; cell division among male embryos occurs more rapidly than in female embryos (Mittwoch U, 1993) and, in humans, boys grow more rapidly than girls from the earliest stages of gestation (Eriksson J G et al., 2010). These differences may start as early as the blastocyst stage in bovines: one third of genes showed sex differences in gene expression (Bermejo-Alvarez P et al., 2008, 2009). Gene expression analysis either for candidate genes or at the genome-wide level show that both the trajectories under basal conditions and those modulating responses differ between the sexes (Gabory A, Attig L, Junien C, 2009). Analysis of genes involved in amino acid transport and metabolism identified sex differences both in average placental gene expression between male and female and in the relationships between placental gene expression and maternal factors (Sturmey R G et al., 2010). Ontological analysis of such data suggests a higher global transcriptionnal level in females and greater protein metabolism levels in males. Specifically, global glucose metabolism and pentose-phosphate pathway activity are twice and four times greater in bovine male vs. female blastocysts respectively, with similar metabolic differences being seen for human embryos at the same stages (Bermejo-Alvarez P et al., 2011). At birth, placental weights and FPI (fetus-to-placenta weight ratio index, reflecting placental efficiency), tend to be greater in boys than girls (Lampl M et al., 2010).

In placentas of female fetuses but not in those of males, an activation of signaling from inflammation via NFκB1 and miR-210 leading to mitochondrial dysfunction was observed. These data suggested that primary trophoblasts derived from placentas of female fetuses showed higher sensitivity to inflammatory stress compared with placentas of males. Moreover, they evidenced the essential role of maternal inflammatory status in regulation of placental mitochondrial metabolism and identify miR-210 as a central component of this fetal sex-biased metabolic regulatory mechanism. Given the importance of genomic imprinting in the placenta, this provides new clues for further investigations of sexual dimorphism in the placenta. This sex difference in epigenome likely accounts for aspects of the reported sex differences in gene and protein expression in the placenta. Global gene changes in the human placenta have been analyzed by Sood et al. (2006). The study clearly defined sex specific differences in placental gene expression not limited to just X and Y linked genes but also autosomal genes related to immune pathways including JAK1, IL2RB, Clusterin, LTBP, CXCL1 and IL1RL1 and TNF receptor expressed at higher levels in female placentae than male placentae. Analysis of genes involved in amino acid transport and metabolism identified gender differences both in average placental gene expression between males and females and in the relationships between placental gene expression and maternal factors, suggesting a higher global transcriptional level in females and greater protein metabolism levels in males (Sturmey R G et al., 2010).

In normal pregnancies, maternal microvascular vasodilatation, which is induced by placental corticotrophin-releasing hormone, is greater in pregnant women carrying male fetuses than in those carrying female fetuses (Clifton V L et al., 2012). Nonetheless, unequal gene expression by the sex chromosomes has an impact much earlier, beginning at conception, and may set the context for events in later life (Davies W, Wilkinson L S, 2006; Al-Khan A et al., 2011). Sex-linked genes and sex hormones may work together to yield similar differences in physiology between the sexes in brain. For instance, immune responses and cytokine production, or sex-linked genes like the androgen receptor, or Y-linked genes may exhibit sex differences because they can be influenced differently by steroid hormones (Xu J, Disteche C M, 2006).

Even before implantation and the initiation of adrenal and gonad development, transcriptional sexual dimorphism is present in various species (Bermejo-Alvarez P et al., 2011). For example, in bovine blastocysts, sex determines the expression levels of one-third of all actively expressed genes (Bermejo-Alvarez P et al., 2010). Sexual dimorphism has also been observed in embryonic cells isolated from mice at E10.5. These cells responded differently to dietary stressors even before the production of fetal sex hormones (Penaloza C et al., 2009). In the mouse, detailed studies on sex chromosomal contribution to placental growth have been reported (Ishikawa H et al., 2003).

Yeganegi et al. (Yeganegi M et al., 2009) have reported that male placentae have higher toll-like receptor-4 (TLR-4) expression and a more enhanced endotoxin induced tumor necrosis factor (TNF)-α response relative to placentae from females. Since a greater population of placental macrophages has been identified in males relative to females of normal pregnancies, the enhanced TNF-α response may be derived from a sex difference in immune cell populations.

Some genes on the X-chromosome are imprinted: their expression is monoallelic, depending on the parental origin of the allele. Recently, three genes have been described as imprinted and expressed from the paternal X allele: Fthl17, Rhox5 and Bex1. These genes are expressed predominantly in female (Kobayashi S et al., 2010). In addition to unequal expression of X-linked genes, the small number of expressed genes present on the Y chromosome (and therefore only expressed in males) may be involved. In humans 29 genes are conserved in the pseudoautosomal regions (PARs) of the X- and Y-chromosomes (Ross M T et al., 2005). The non-recombining, male-specific Y region contains about 27 protein-coding genes (Skaletsky H et al., 2003).

In study of Gonzalez T L et al. (2018) RNA-sequencing was performed to characterize the transcriptome of 39 first trimester human placentas using chorionic villi following genetic testing (17 females, 22 males). Gene enrichment analysis was performed to find enriched canonical pathways and gene ontologies in the first trimester. DESeq2 was used to find sexually dimorphic gene expression. Patient demographics were analyzed for sex differences in fetal weight at time of chorionic villus sampling and birth. RNA-sequencing analyses detected 14,250 expressed genes, with chromosome 19 contributing the greatest proportion (973/2852, 34.1% of chromosome 19 genes) and Y chromosome contributing the least (16/568, 2.8%). Several placenta-enriched genes as well as histone-coding genes were identified to be unique to the first trimester and common to both sexes. Further, authors identified 58 genes with significantly different expression between males and females: 25 X-linked, 15 Y-linked, and 18 autosomal genes. Genes that escape X inactivation were highly represented (59.1%) among X-linked genes upregulated in females. Many genes differentially expressed by sex consisted of X/Y gene pairs, suggesting that dosage compensation plays a role in sex differences. These X/Y pairs had roles in parallel, ancient canonical pathways important for eukaryotic cell growth and survival: chromatin modification, transcription, splicing, and translation.

In humans and other placental mammals, the fertilized egg gives rise to both the fetus and the placenta. Placentation in the first trimester can impact fetal growth, and abnormal placentation can lead to more pronounced effects complicating pregnancy including intrauterine growth restriction (IUGR) which results in very low birth weight infants, a sexually dimorphic outcome (Redman C W, Sargent I L, 2005; Pijnenborg R, Vercruysse L, Hanssens M., 2006; Wang E T et al., 2017; Sundheimer L W, Pisarska M D, 2017). The process of placentation occurs throughout the first trimester of pregnancy, whereby the outer cells of the blastocyst (the trophoblast cells) invade the maternal tissue and develop into the placenta. It is a highly regulated state of active cell proliferation, cell migration, and cell differentiation (Kroener L, Wang E T, Pisarska M D, 2016). To sustain this growth, placentation is in a high state of transcriptional activity, as evidenced by its marked hypomethylated state (Ball M P et al., 2009; Chu T et al., 2011; Schroeder D I et al., 2013; Bianco-Miotto T et al., 2016). Placentation requires multiple factors, including maternal immune tolerance, various growth factors, fetal-maternal communication via biochemical signaling, and a receptive maternal decidua that allows extravillous trophoblast cells to invade the maternal circulatory system and access maternal nutrients throughout pregnancy (Roberts C T, 2010; Segars J H et al., 1989; O'Tierney-Ginn P F, Lash G E, 2014; Whitley G S J, Cartwright J E., 2009).

Evolutionary constraints may be responsible for the presence of placental genes on the X chromosome that are co-expressed in brain and testis (Hemberger M, 2002). In human term placentas, Sood et al. (2006) have shown that many of the sex-correlated genes are located on the sex chromosomes, but that some are autosomal (Sood R et al., 2006). In addition, X- and Y-linked genes may modulate the expression of different sets of autosomal genes, leading to differences in physiological trajectories between males and females (Gabory A, Attig L, Junien C, 2009). Thus, both the trajectories under basal conditions and those modulating responses differ between the sexes.

Upstream analysis in IPA identified the top 40 gene regulators upstream of the top 25% expressed genes. Most upstream regulators were associated with either essential cell regulation, cell growth, or hormonal signaling. Transcriptional regulators such as MYC, p53, MYCN, and HNF4A were among the most significant upstream regulators. Regulators involved in cell growth include rapamycin-insensitive companion of mTOR (RICTOR), transforming growth factor beta 1 (TGFB1), D-glucose, epidermal growth factor receptor (EGFR), as well as epidermal growth factor (EGF). In addition, hormones such as beta-estradiol, its receptor (ESR1), as well as the progesterone receptor (PGR) were also significant upstream regulators. Together, these upstream regulators control essential cell regulation, cell growth, and hormonal signaling, consistent with the canonical pathways identified (https://bsd.biomedcentral.com/articles/10.1186/s13293-018-0165-y#MOESM3).

The epigenetic landscape required for placenta development has been described (Hemberger M, 2007). The sex of the placenta and the environment have an influence on its epigenomes, and hence on the epigenomes of the developing fetus. In all adult tissues examined to date, including the gonads and brain, the expression of many genes is modulated in a sex-specific manner (Kang H J et al., 2012; Yang X et al., 2006). Chromatin structure and epigenetic marks differ between male and female samples in brain (Qureshi I A, Mehler M F, 2010; McCarthy M M et al., 2009). The adult liver is the organ in which these aspects have been best characterized, with genome-wide DNaseI-hypersensitive sites and sex-specific gene expression detected (Waxman D J, Holloway M G, 2009; Ling G et al., 2010; van Nas A et al., 2009; Wauthier V et al., 2010).

Sexually dimorphic patterns of gene expression have recently been reported for individual genes in placentas from humans and rodents (Gabory A, Attig L, Junien C, 2009). Sex differences have been observed in the mRNA levels of housekeeping genes and of commonly used reference genes in human placenta, in a variety of mouse somatic and extra-embryonic tissues, as well as in the preimplantation blastocyst and blastocyst-derived embryonic stem cells (Cleal J K et al., 2010; Lucas E S et al., 2011). Few groups have studied global sexual dimorphism in the placenta with microarrays, focusing in particular on the impact of maternal diet, asthma or stress on placental gene expression, through systemic investigations of the relationship between diet and the expression of sexually dimorphic genes. These transcriptomic analyses showed that basal gene expression levels were sexually dimorphic in whole placentas (Gabory A et al., 2012). In addition to sex-specific differences in the endocrine and immune systems (Hochberg Z et al., 2011; Invernizzi P et al., 2009), sex-specific genetic architecture (Invernizzi P et al., 2009) also influences placental growth and specific functions (Hemberger M et al., 2001; Ober C, Loisel D A, Gilad Y, 2008).

To validate this potentially novel first trimester placenta expression, we cross-referenced RNA-sequencing data of term placentas (NCBI GEO Accession GSE73016) and found that EBI3 and HISTIH2BO were expressed in term placenta (Yabe S et al., 2016). We also compared the top 175 protein-coding genes to a microarray study that compared early first trimester placenta (45-59 days) versus C-section delivered term placentas (NCBI GEO Accession GSE9984) and found 23 genes that are differentially expressed between early pregnancy and delivery. Of the top 175 genes, 5 were significantly upregulated in first trimester (CD24, COL6A2, ENO1, HMGA1, KRT7) and 18 genes were significantly upregulated in term placenta (ADAM12, CYP19A1, EBI3, FBLNI, GDF1S, HSPB8, KISS1, PAPPA, PAPPA2, PSG1, PSG3, PSG4, PSG6, PSG9, S100A9, S100P, SDC1, SLC2A1) (Mikheev A M et al., 2008). The XAGE2, XAGE3, CGB family, and histone-encoding genes were not identified as gestationally different. Overall, there remained 11 histone-encoding genes highly expressed in late first trimester placenta that showed no UniGene-documented placenta expression nor expression in term placenta (Saben J et al., 2014), suggesting they are unique to first trimester placenta and potentially critical for early placentation.

At week 11, there were 30 differentially expressed genes between male and female chorionic villus samples. Three new autosome genes (STAT6, SLC4A1, LAMB3) were significantly upregulated in males. The remaining 17 sex-linked differentially expressed genes were consistent with the larger 39 sample analysis: 4 female-upregulated X-linked genes (XIST, KDM6A, EIF1AX, and NUDT10) and 13 male-upregulated Y-linked genes all had false discovery rate <0.05 in the 39-sample analysis. At week 13, there were 20 differentially expressed genes between males and females (https://bsd.biomedcentral.com/articles/10.1186/si3293-018-0165-y#MOESM3). Two new autosome genes were male-upregulated (SERPINB7 and PLTP). Sex-linked differentially expressed genes were consistent with 39 sample results: female-upregulated X-linked genes (XIST and KDMSC), male-upregulated X-linked genes (ARMCX3 and ARMCX3), and 14 Y-linked genes (all except PSMA6P1). Eleven histone-encoding genes highly expressed in first trimester placenta were not found in UniGene's expressed sequence tag profile for placenta, have not been previously described in term placenta, and are also not among highly expressed genes in term placenta transcriptome studies (Mikheev A M et al., 2008; Saben J et al., 2014), showing that the first trimester placenta transcriptome is different from term placenta. Histones are DNA-binding proteins responsible for packaging DNA into nucleosomes, and differences in histones and histone modifications are associated with differences in DNA accessibility for transcription. The high expression of these histone-encoding genes in the first trimester placenta, but not later in gestation, suggests that the first trimester chromatin structure is unique and functions to maintain a high transcriptionally active state. Eight of these histone genes (HIST1H1B, HIST1H1E, HISTIH3B, HISTIH3C, HISTIH3F, HISTIH4B, HISTIH4D, and HISTIH4F) had very limited tissue expression in non-placental tissue, primarily appearing in embryonic, vascular, skin, and connective tissue.

There were eight X-linked genes that were upregulated in females only in the late first trimester placentas: BRCC3, CHM, HDAC8, LINC00630, MIR6895, OFDI, RP13-36G14.4, and YIPF6. These genes play important roles in transcriptional regulation, DNA damage response, and vesicle-mediated transport, suggesting biological processes that may be sex-biased and critical for early placenta development. LINC00630 promotes cell proliferation by promoting the protein stability of histone deacetylate HDAC1 (Mao G, Jin H, Wu L, 2017). Non-coding gene MIR6895 is antisense to histone demethylase-encoding KDMSC, and thus may also regulate placental chromatin status. Among males, 13 Y-linked genes were consistent with that in the adult tissues, but pseudogenes ANOS2P and PSMA6P1 exhibited male-upregulation only in the first trimester placenta. Similar to other pseudogenes, they may act as miRNA decoys which may be important in early development (Milligan M J, Lipovich L., 2014; Pink R C et al., 2011).

Sexually dimorphic genes are likely functionally relevant for placenta biology. Females had a greater number of upregulated genes encoding DNA binding proteins, including genes associated with chromatin modification (including X inactivation) and genes encoding components of centrioles and cohesin complexes (OFD1 and SMC1A, respectively) important for segregation of replicated DNA during cell division (Lopes C A et al., 2011; Gregson H C et al., 2002). Metabolism-associated upregulated genes in males were primarily involved in response to nutrient deficit. MTRNR2L8 (1.61-fold upregulated in males) encodes Humanin-like 8, a small peptide homologous and sometimes identical (due to a polymorphic site) to the mitochondrially encoded Humanin peptide which promotes cell survival in ATP-deficient environments (Bodzioch M et al., 2009; Kin T et al., 2006). Humanin promotes insulin sensitivity which may contribute to increased fetal growth seen in males (Muzumdar R H et al., 2009). HMGCS2 (1.60-fold upregulated in males) encodes an enzyme that promotes autophagy by catalyzing the first step in ketogenesis, a pathway that derives energy from lipids when carbohydrates are depleted (Hu L T et al., 2017). This is consistent with previous pregnancy studies that find greater risk for nutrient deficit in males (Barker D J et al., 2012). In contrast, metabolism-associated genes upregulated in females are involved in post-transcriptional modification of RNA (PUDP) and hormone biosynthesis (STS) (Preumont A et al., 2010; Reed M J et al., 2005). Cell adhesion, ciliogenesis, and cell-cell communication genes (OFD1, OSBL3, PCDH11Y, TBC1D32) were also differentially expressed, suggesting sex differences in how placenta cells interact with their environment (Lehto M et al., 2008; Berx G, van Roy F., 2009; Adly N et al., 2014). ITGB8 (encodes integrin-08) promotes tumor angiogenesis and invasiveness in glioblastoma (Tchaicha J H et al., 2011), functions necessary for normal first trimester development when placental cells invade maternal tissue and access maternal blood.

Gestational age-specific sex differences exist, even between 11- and 13-weeks' gestation. It was found that a subset of Y-linked genes is consistently expressed in late first trimester placenta regardless of gestational age and into adulthood, whereas X-linked genes and autosome genes were more variable. However, X-linked genes that affect chromatin modification (XIST, KDM6A, KDMSC) are conserved throughout gestation and into adulthood (Mele M et al., 2015).

Gonzalez T L et al. (2018) identified a group of 11 histone-encoding genes which were highly expressed in first trimester placenta, but have not been previously reported in term placenta, suggesting they may have temporally specific functions important for early pregnancy regardless of sex. When examining our cohort of patients for sex differences, authors found 58 significantly differentially expressed genes with an overrepresentation of genes known to escape X inactivation. Many of the sexually dimorphic genes fell into a group of X/Y gene pairs, suggesting that dosage compensation plays a role in sex differences.

Female and male placentas have different strategies to optimize health: actually, the two sexes present different optimal transcriptomes that may affect fetal growth and later health or disease. The male strategy for responding to an adverse maternal environment is a minimalist approach: few genes, proteins or functional changes are involved in the placenta, which ultimately ensures continued growth in a less than optimal maternal environment. This specific male response is associated with a greater risk of intrauterine growth restriction, preterm delivery or death in utero if another adverse event occurs during the pregnancy. The female placenta responds to an adverse maternal environment with multiple placental gene and protein changes that result in a decrease in growth without growth restriction. Thus, female adjustments in placental function and growth ensure survival in the presence of another adverse event which may further compromise nutrient or oxygen supply (Di Renzo G. C. et al., 2015).

The results obtained by us for the determination of biologically active proteins in the conditioned medium of the XX-placenta and XY-placenta explants fully consistent with the literature data on sexual dimorphism of the placenta in the first trimester of pregnancy. Half of the 10 studied growth factors and cytokines in conditioned medium of XY- and XX-placenta explants differed significantly and authentically: XY-placental explants produced 22 times more HGF, 2 times more SCF, 1.3 times more IGF-1, and almost 3 times more bFGF than XX-placental explants. In turn, XX-placental explants produced 3.5 times more TGF-beta 1 than XY-placental explants

Since all 10 biologically active proteins (EGF, FGF-7, HGF, TNF-alpha, IGF-1, VEGF-A, SCF, TGF-beta 1, ANGPT1, bFGF) studied by us are involved in the processes of physiological regeneration of skin and hair (Chen D. et al., 2019; Imokawa G., 2004; Shan-Chang Chueh at al., 2013), we believe that the combinations of growth factors and cytokines obtained by cultivating XX- or XY-placental explants at the time of the formation of provisional organs and fetal tissues completion are unique and have a high regenerative capacity potential when used in cosmetics to prevent skin and hair aging.

### REFERENCES

Adly N et al. (2014) “Ciliary genes TBC1D32/C6orf170 and SCLT1 are mutated in patients with OFD type IX” Hum Mutat 35(1): 36-40.
Al-Khan A et al. (2011) “Meeting 2010 Workshops Report II: placental pathology; trophoblast invasion; fetal sex; parasites and the placenta; decidua and embryonic or fetal loss; trophoblast differentiation and syncytialisation” Placenta 32(2): S90-S99.
Avissar-Whiting M et al. (2010) “Bisphenol A exposure leads to specific microRNA alterations in placental cells” Reprod Toxicol 29: 401-6.
Ball M P et al. (2009) “Targeted and genome-scale strategies reveal gene-body methylation signatures in human cells” Nat Biotechnol 27(4): 361-8.
Barker D J et al. (2012) “Resource allocation in utero and health in later life” Placenta 33 (Suppl 2): e30-4.
Bermejo-Alvarez P et al. (2008) “Epigenetic differences between male and female bovine blastocysts produced in vitro” Physiol Genomics 32: 264-72.
Bermejo-Alvarez P et al. (2009) “Micro-array analysis reveals that one third of the genes actively expressed are differentially expressed between male and female bovine blastocysts” Biol Reprod 81.
Bermejo-Alvarez P et al. (2010) “Sex determines the expression level of one third of the actively expressed genes in bovine blastocysts” Proc Natl Acad Sci USA 107: 3394-9. doi: 10.1073/pnas.0913843107.
Bermejo-Alvarez P et al. (2011) “Transcriptional sexual dimorphism during preimplantation embryo development and its consequences for developmental competence and adult health and disease” Reproduction 141: 563-70. doi: 10.1530/REP-10-0482.
Berx G, van Roy F. (2009) “Involvement of members of the cadherin superfamily in cancer” Cold Spring Harb Perspect Biol 1(6): a003129.
Bianco-Miotto T et al. (2016) “Recent progress towards understanding the role of DNA methylation in human placental development” Reproduction 152(1): R23-30.
Bodzioch M et al. (2009) “Evidence for potential functionality of nuclearly-encoded human in isoforms” Genomics 94(4): 247-56.
Cedar H, Bergman Y. (2009) “Linking DNA methylation and histone modification: patterns and paradigms” Nat Rev Genet 10: 295-304.
Chen D. et al (2019) “Bioactive Molecules for Skin Repair and Regeneration: Progress and Perspectives” Stem Cell Int Article ID6789823 https://doi.org/10.1155/2019/6789823
Chu T et al. (2011) “Structural and regulatory characterization of the placental epigenome at its maternal interface” PLoS One 6(2): e14723.
Cleal J K (2010) “Sex differences in the mRNA levels of housekeeping genes in human placenta” Placenta 31: 556-7. doi: 10.1016/j.placenta.2010.03.006.
Clifton V et al. (2009) “Sex specific function of the human placenta: implication for fetal growth and survival” Reprod Fertil Dev 21: 9. doi: 10.1071/SRB09Abs036.
Clifton V L et al. (2012) “Review: the feto-placental unit, pregnancy pathology and impact on long term maternal health” Placenta 33: S37-S41.
Clifton V L, Vanderlelie J, Perkins A V. (2005) “Increased anti-oxidant enzyme activity and biological oxidation in placentae of pregnancies complicated by maternal asthma” Placenta 26: 773-9.
Clifton V L. (2005) “Sexually dimorphic effects of maternal asthma during pregnancy on placental glucocorticoid metabolism and fetal growth” Cell Tissue Res 322: 63-71. doi: 10.1007/s00441-005-1117-5.
Clifton V L. (2010) “Review: sex and the human placenta: mediating differential strategies of fetal growth and survival” Placenta 31: S33-S39.
Davies W, Wilkinson L S. (2006) “It is not all hormones: alternative explanations for sexual differentiation of the brain” Brain Res 1126: 36-45. doi: 10.1016/j.brainres.2006.09.105.
de Castro F, Seal R, Maggi R. (2016) “ANOS1: a unified nomenclature for Kallmann syndrome 1 gene (KAL1) and anosmin-1” Brief Funct Genomics elw037(2041-2657)
Dhurat R, Saraogi P. (2009) “Hair Evaluation Methods: Merits and Demerits” Int J Trichology 1(2): 108-19. doi: 10.4103/0974-7753.58553
Di Renzo G. C. et al. (2015) “Is there a sex of the placenta?” J Pediatr Neonat Individ Med 4(2).
Di Renzo G C et al. (2007) “Does fetal sex affect pregnancy outcome?” Gend Med 4: 19-30.
Engel P J et al. (2008) “Male sex and pre-existing diabetes are independent risk factors for stillbirth” Aust New Zealand J Obstet Gynaecol 48: 375-83.
Ennen et al. (2014) “Studies on the Skin Compatibility of Cosmetics Involving Human Subjects—Study Portfolio and Test Strategy” IFSCC Magazine 1: 19-23.
Enquobahrie D A et al. (2011) “Placental microRNA expression in pregnancies complicated by preeclampsia” Am J Obstet Gynecol 204: 178.e12-21.
Eriksson J G et al. (2010) “Boys live dangerously in the womb” Am J Hum Biol 22: 330-5. doi: 10.1002/ajhb.20995.
Escoffier C et al. (1989) “Age-related mechanical properties of human skin: an in vivo study” J Invest Dermatol 93:353-7. doi: 10.1111/1523-1747.ep12280259.
Gabory A et al. (2012) “Maternal diets trigger sex-specific divergent trajectories of gene expression and epigenetic systems in mouse placenta” PLoS One 7: e47986. doi: 10.1371/journal.pone.0047986.
Gabory A et al. (2013) “Placental contribution to the origins of sexual dimorphism in health and diseases: sex chromosomes and epigenetics” Biol Sex Differ 4(1): 5.
Gabory A, Attig L, Junien C (2009) “Sexual dimorphism in environmental epigenetic programming” Mol Cell Endocrinol 25: 8-18.
Gabory A. et al. (2013) “Placental contribution to the origins of sexual dimorphism in health and diseases: sex chromosomes and epigenetics” Biol Sex Differ 4: 5. doi: 10.1186/2042-6410-4-5
Gonzalez T L et al. (2018) “Sex differences in the late first trimester human placenta transcriptome” Biology of Sex Differences 9: 4.).
Gregson H C et al. (2002) “Localization of human SMC1 protein at kinetochores” Chromosom Res 10(4): 267-77.
Gu Y et al. (2013) “Differential miRNA expression profiles between the first and third trimester human placentas” Am J Physiol Endocrinol Metab 304(8): E836-43.
Guil S, Esteller M. (2009) “DNA methylomes, histone codes and miRNAs: tying it all together” Int J Biochem Cell Biol 41: 87-95.
Han L et al. (2007) “DNA methylation regulates MicroRNA expression” Cancer Biol Ther 6: 1290-4.
Hemberger M et al. (2001) “Genetic and developmental analysis of X-inactivation in interspecific hybrid mice suggests a role for the Y chromosome in placental dysplasia” Genetics 157: 341-8.
Hemberger M. (2002) “The role of the X chromosome in mammalian extra embryonic development” Cytogenet Genome Res 99: 210-7. doi: 10.1159/000071595.
Hemberger M. (2007) “Epigenetic landscape required for placental development” Cell Mol Life Sci 64: 2422-36. doi: 10.1007/s00018-007-7113-z.
Hochberg Z et al. (2011) “Child health, developmental plasticity, and epigenetic programming” Endocr Rev 32: 159-224. doi: 10.1210/er.2009-0039.
Hu L T et al. (2017) “HMGCS2 promotes autophagic degradation of the amyloid-beta precursor protein through ketone body-mediated mechanisms” Biochem Biophys Res Commun 486(2): 492-8.
Imokawa G. (2004) “Autocrine and Paracrine Regulation of Melanocytes in Human Skin and in Pigmentary Disorders” Pigment Cell Research 17(2): 96-110 DOI:10.1111/j.1600-0749.2003.00126.x
Inui M, Martello G, Piccolo S. (2010) “MicroRNA control of signal transduction” Nat Rev Mol Cell Biol 11(4): 252-63.
Ishikawa H et al. (2003) “Effects of sex chromosome dosage on placental size in mice” Biol Reprod 69: 483-8. doi: 10.1095/biolreprod.102.012641.
Jenuwein T, Allis C D. (2001) “Translating the histone code” Science 293: 1074-80.
John R, Hemberger M (2012) “A placenta for life” Reprod Biomed Online 25: 5-11. doi: 10.1016/j.rbmo.2012.03.018.
Kang H J et al. (2012) “Spatio-temporal transcriptome of the human brain” Nature 478: 483-9.
Kin T et al. (2006) “Humanin expression in skeletal muscles of patients with chronic progressive external ophthalmoplegia” J Hum Genet 51(6): 555-8.
Kobayashi S et al. (2010) “The X-linked imprinted gene family Fthl17 shows predominantly female expression following the two-cell stage in mouse embryos” Nucleic Acids Res 38: 3672-81. doi: 10.1093/nar/gkg113.
Kroener L, Wang E T, Pisarska M D. (2016) “Predisposing factors to abnormal first trimester placentation and the impact on fetal outcomes” Semin Reprod Med 34(1): 27-35.
Lahti J et al. (2009) “Early-life origins of schizotypal traits in adulthood” Br J Psychiatry 195: 132-137. doi: 10.1192/bjp.bp.108.054387.
Lampl M et al. (2010) “Sex differences in fetal growth responses to maternal height and weight” Am J Hum Biol 22: 431-43.
Langley R G B et al. (2013) “The 5-point Investigator's Global Assessment (IGA) Scale: A modified tool for evaluating plaque psoriasis severity in clinical trials” J Dermatolog Treat 26(1): 23-31. doi: 10.3109/09546634.2013.865009.
Lehto M et al. (2008) “The R-Ras interaction partner ORP3 regulates cell adhesion” J Cell Sci 121 (Pt 5): 695-705.
Ling G et al. (2010) “Unbiased, genome-wide in vivo mapping of transcriptional regulatory elements reveals sex differences in chromatin structure associated with sex-specific liver gene expression” Mol Cell Biol 30: 5531-44. doi: 10.1128/MCB.00601-10.
Lopes C A et al. (2011) “Centriolar satellites are assembly points for proteins implicated in human ciliopathies, including oral-facial-digital syndrome 1” J Cell Sci 124 (Pt 4): 600-12.
Lucas E S et al. (2011) “Tissue-specific selection of reference genes is required for expression studies in the mouse model of maternal protein undernutrition” Theriogenology 76: 558-69. doi: 10.1016/j.theriogenology.2011.03.008.
Maccani M A et al. (2010) “Maternal cigarette smoking during pregnancy is associated with down regulation of miR-16, miR-21, and miR-146a in the placenta” Epigenetics 5: 583-9.
Maccani M A, Padbury J F, Marsit C J. (2011) “miR-16 and miR-21 expression in the placenta is associated with fetal growth” PLoS One 6: e21210.
Mao G, Jin H, Wu L. (2017) “DDX23-Linc00630-HDAC1 axis activates the notch pathway to promote metastasis” Oncotarget 8(24): 38937-49.
Mao J et al. (2010) “Contrasting effects of different maternal diets on sexually dimorphic gene expression in the murine placenta” Proc Natl Acad Sci USA 107: 5557-62. doi: 10.1073/pnas.1000440107.
McCarthy M M et al. (2009) “The epigenetics of sex differences in the brain” J Neurosci 29: 12815-23. doi: 10.1523/JNEUROSCI.3331-09.2009.
Mele M et al. (2015) “The human transcriptome across tissues and individuals” Science 348(6235): 660-5.
Migeon B R, Axelman J, Jeppesen P. (2005) “Differential X reactivation in human placental cells: implications for reversal of X inactivation” Am J Hum Genet 77: 355-64.
Mikheev A M et al. (2008) “Profiling gene expression in human placentae of different gestational ages: an OPRU network and U W SCOR study” Reprod Sci 15(9): 866-77.
Milligan M J, Lipovich L. (2014) “Pseudogene-derived lncRNAs: emerging regulators of gene expression” Front Genet 5: 476.
Mittwoch U. (1993) “Blastocysts prepare for the race to be male” Hum Reprod 8: 1550-5.
Morales-Prieto D M et al. (2012) “MicroRNA expression profiles of trophoblastic cells” Placenta 33(9): 725-34.
Moritz K M et al. (2010) “Review: sex specific programming: a critical role for the renal renin-angiotensin system” Placenta 31: S40-S46.
Muzumdar R H et al. (2009) “Humanin: a novel central regulator of peripheral insulin action” PLoS One 4(7): e6334.
Ober C, Loisel D A, Gilad Y. (2008) “Sex-specific genetic architecture of human disease” Nat Rev Genet 9: 911-22. doi: 10.1038/nrg2415.
O'Tierney-Ginn P F, Lash G E. (2014) “Beyond pregnancy: modulation of trophoblast invasion and its consequences for fetal growth and long-term children's health” J Reprod Immunol 104-105: 37-42.
Penaloza C et al. (2009) “Sex of the cell dictates its response: differential gene expression and sensitivity to cell death inducing stress in male and female cells” FASEB J 23:1869-79. doi: 10.1096/fj.08-119388.
Pijnenborg R, Vercruysse L, Hanssens M. (2006) “The uterine spiral arteries in human pregnancy: facts and controversies” Placenta 27(9-10): 939-58.
Pink R C et al. (2011) “Pseudogenes: pseudo-functional or key regulators in health and disease?” RNA 17(5): 792-8.
Preumont A et al. (2010) “HDHD1, which is often deleted in X-linked ichthyosis, encodes a pseudouridine-5′-phosphatase” Biochem J 431(2): 237-44.
Qureshi I A, Mehler M F. (2010) “Genetic and epigenetic underpinnings of sex differences in the brain and in neurological and psychiatric disease susceptibility” Prog Brain Res 186: 77-95.
Redman C W, Sargent I L. (2005) “Latest advances in understanding preeclampsia” Science 308(5728): 1592-4.
Reed M J et al. (2005) “Steroid sulfatase: molecular biology, regulation, and inhibition” Endocr Rev 26(2): 171-202.
Roberts C T. (2010) “IFPA award in placentology lecture: complicated interactions between genes and the environment in placentation, pregnancy outcome and long-term health” Placenta 31: S47-53.
Roseboom T J et al. (2011) “Effects of famine on placental size and efficiency” Placenta 32: 395-399. doi: 10.1016/j.placenta.2011.03.001.
Ross M T et al. (2005) “The DNA sequence of the human X chromosome” Nature 434: 325-37. doi: 10.1038/nature03440.
Saben J et al. (2014) “A comprehensive analysis of the human placenta transcriptome” Placenta 35(2): 125-31.
Schroeder D I et al. (2013) “The human placenta methylome” Proc Natl Acad Sci USA 110(15): 6037-42.
Segars J H et al. (1989) “The human blastocyst produces a soluble factor(s) that interferes with lymphocyte proliferation” Fertil Steril 52(3): 381-7.
Shan-Chang Chueh at al. (2013) “Therapeutic strategy for hair regeneration: Hair cycle activation, niche environment modulation, wound-induced follicle neogenesis and stem cell engineering” Expert Opin Biol Ther 13(3): 377-91. doi:10.1517/14712598.2013.739601
Shuster S, Black M M, McVitie E (1975) “The influence of age and sex on skin thickness, skin collagen and density” Br J Dermatol 93: 639-43. doi: 10.1111/j.1365-2133.1975.tb05l13.x.)
Skaletsky H et al. (2003) “The male-specific region of the human Y chromosome is a mosaic of discrete sequence classes” Nature 423: 825-37. doi: 10.1038/nature01722.
Sood R et al. (2006) “Gene expression patterns in human placenta” Proc Natl Acad Sci USA 103: 5478-83. doi: 10.1073/pnas.0508035103.
Stevenson D K et al. (2000) “Sex differences in outcomes of very low birthweight infants: the newborn male disadvantage” Arch Dis Child Fetal Neonatal Ed 83: F182-5.
Sturmey R G et al. (2010) “Amino acid metabolism of bovine blastocysts: a biomarker of sex and viability” Mol Reprod Dev 77: 285-96.
Sundheimer L W, Pisarska M D. (2017) “Abnormal placentation associated with infertility as a marker of overall health” Semin Reprod Med 35(3): 205-16.
Tchaicha J H et al. (2011) “Glioblastoma angiogenesis and tumor cell invasiveness are differentially regulated by beta8 integrin” Cancer Res 71(20): 6371-81.
Thornburg K L, O'Tierney P F, Louey S. (2010) “Review: the placenta is a programming agent for cardiovascular disease” Placenta 31: S54-S59.
Tuddenham L et al. (2006) “The cartilage specific microRNA-140 targets histone deacetylase 4 in mouse cells” FEBS Lett 580: 4214-7.
van Nas A et al. (2009) “Elucidating the role of gonadal hormones in sexually dimorphic gene coexpression networks” Endocrinology 150: 1235-49.
Varambally S et al. (2008) “Genomic loss of microRNA-101 leads to overexpression of histone methyltransferase EZH2 in cancer” Science 322: 1695-9.
Varani J et al. (2001) “Inhibition of type I procollagen synthesis by damaged collagen in photoaged skin and by collagenase-degraded collagen in vitro” Am J Pathol 158: 931-42. doi: 10.1016/S0002-9440(10)64040-0.
Vatten L J, Skjaerven R. (2004) “Offspring sex and pregnancy outcome by length of gestation” Early Hum Dev 76: 47-54.
Wang E T et al. (2017) “Abnormal implantation after fresh and frozen in vitro fertilization cycles” Fertil Steril 107(5): 1153-8.
Wauthier V et al. (2010) “Intrinsic sex differences in the early growth hormone responsiveness of sex-specific genes in mouse liver” Mol Endocrinol 24: 667-678. doi: 10.1210/me.2009-0454.
Waxman D J, Holloway M G. (2009) “Centennial perspective: sex differences in the expression of hepatic drug metabolizing enzymes” Mol Pharmacol 76(2): 215-28. doi: 10.1124/mol.109.056705.
Whitley G S J, Cartwright J E. (2009) “Trophoblast-mediated spiral artery remodelling: a role for apoptosis” J Anat 215(1): 21-6.
Wong C F, Tellam R L. (2008) “MicroRNA-26a targets the histone methyltransferase Enhancer of Zeste homolog 2 during myogenesis” J Biol Chem 283: 9836-43.
Wu L et al. (2010) “DNA methylation mediated by a microRNA pathway” Mol Cell 38: 465-75.
Xu J, Disteche C M. (2006) “Sex differences in brain expression of X- and Y-linked genes” Brain Res 1126: 50-55. doi: 10.1016/j.brainres.2006.08.049.
Yabe S et al. (2016) “Comparison of syncytiotrophoblast generated from human embryonic stem cells and from term placentas” Proc Natl Acad Sci USA 113(19): E2598-607.
Yang X et al. (2006) “Tissue-specific expression and regulation of sexually dimorphic genes in mice” Genome Res 16: 995-1004. doi: 10.1101/gr.5217506.
Yeganegi M et al. (2009) “Effect of Lactobacillus rhamnosus GR-1 supernatant and fetal sex on lipopolysaccharide-induced cytokine and prostaglandin-regulating enzymes in human placental trophoblast cells: implications for treatment of bacterial vaginosis and prevention of preterm labor” Am J Obstet Gynecol 200(5): 532.el-8.

1: Anti-ageing cosmetic compositions for men and woman comprising bioactive protein, which are obtained by the method of cultivation of explants XX-placenta (for women's cosmetics) or XY-placenta (for men's cosmetics) comprises the steps of: i. Making explants of XX-placenta or XY-placenta in gestation term not less 11 weeks, and not more 12 weeks. ii. Cultivation explants of XX-placenta or XY-placenta until the moment of migration and proliferation of cells on an area of not more than 4 cm² around each explant. iii. Filtration of the conditioned medium of XX-placenta or XY-placenta explants and adding a proteolysis inhibitor to the medium. iv. Control of biological safety of the conditioned medium of XX-placenta or XY-placenta explants (microbial, viral and fungal contamination). v. Controlling the concentration of biologically active proteins in a conditioned medium of XX-placenta or XY-placenta explants. vi. Adding of XX- or XY-placental compositions of biologically active proteins to the ingredients of cosmetic products. vii. Controlling the concentration of biologically active proteins of the XX-placenta or XY-placenta in ready-to-use cosmetic products.

2: The method of making explants of XX-placenta or XY-placenta according to claim 1; wherein explants production performed after medical pregnancy termination in the gestational period 11-12 weeks.

3: The method for obtaining biologically active proteins in a conditioned medium of XX-placenta or XY-placenta explants according to claim 1; wherein cultivation of XX-placenta or XY-placenta explants stops immediately after migration and proliferation of placental cells in an area of up to 4 cm² around each explant.

4: The method as claimed in claim 1, wherein conditioned medium of XX-placenta or XY-placenta explants is cleared of tissue and cells of placental explants by filtration.

5: The method as claimed in claim 1, wherein the degradation of biologically active proteins is prevented by the adding of the proteolysis inhibitor aprotinin to the conditioned medium of XX-placenta or XY-placenta explants.

6: The method as claimed in claim 1, wherein control of biological safety of the conditioned medium of XX-placenta or XY-placenta explants (microbial, viral and fungal contamination) is carried out according to the guidelines for monitoring the biological safety of stem cells and their derivatives.

7: The method as claimed in claim 1, wherein for control the concentrations of biologically active proteins in a conditioned medium of XY-placenta explants perfumed ELISA-test of references levels for: EGF—from 10 to 18 pg/ml; FGF-7—from 74 to 300 pg/ml; HGF—from 941 to 2039 pg/ml; TNF-alpha—from 340 to 521 pg/ml; IGF-1—from 630 to 928 ng/ml; VEGF-A—from 12 to 38 ng/ml; SCF—from 15.5 to 24 ng/ml; TGF-beta 1—from 66.5 to 178 ng/ml; ANGPT1—from 94 to 170 ng/ml; bFGF—from 58 to 97 ng/ml.

8: The method as claimed in claim 1, wherein for control the concentrations of biologically active proteins in a conditioned medium of XX-placenta explants perfumed EFISA-test of references levels for: EGF—from 8 to 18 pg/ml; FGF-7—from 90 to 265 pg/ml; HGF—from 30 to 104 pg/ml; TNF-alpha—from 340 to 546 pg/ml; IGF-1—from 514 to 681 ng/ml; VEGF-A—from 16 to 39 ng/ml; SCF—from 7.5 to 12 ng/ml; TGF-beta 1—from 311 to 550 ng/ml; ANGPT1—from 104 to 156 ng/ml; bFGF—from 14 to 43 ng/ml.

9: The method as claimed in claim 1, wherein adding of XX- or XY-placental compositions of biologically active proteins to the ingredients of cosmetic products, which are compatible with biologically active proteins and do not destroy them.

10: The method as claimed in claim 1, wherein controlled concentrations of biologically active proteins of the XY-placenta in ready-to-use cosmetic products must be not less then for: EGF—1.0 pg/ml; FGF—7-7.4 pg/ml; HGF—94.1 pg/ml; TNF-alpha—34.0 pg/ml; IGF-1—63.0 ng/ml; VEGF-A—1.2 ng/ml; SCF—1.6 ng/ml; TGF-beta 1—6.7 ng/ml; ANGPT1— 9.4 ng/ml; bFGF—5.8 ng/ml.

11: The method as claimed in claim 1, wherein controlled concentrations of biologically active proteins of the XX-placenta in ready-to-use cosmetic products must be not less then for: EGF—0.8 pg/ml; FGF—7-9.0 pg/ml; HGF—3.0 pg/ml; TNF-alpha—34.0 pg/ml; IGF-1—51.4 ng/ml; VEGF-A—1.6 ng/ml; SCF—0.8 ng/ml; TGF-beta 1—31.1 ng/ml; ANGPT1— 10.4 ng/ml; bFGF—1.4 ng/ml.

12: The method as claimed in claim 3, wherein conditioned medium of XX-placenta or XY-placenta explants is cleared of tissue and cells of placental explants by filtration.

13: The method as claimed in claim 4, wherein the degradation of biologically active proteins is prevented by the adding of the proteolysis inhibitor aprotinin to the conditioned medium of XX-placenta or XY-placenta explants.

14: The method as claimed in claim 3, wherein control of biological safety of the conditioned medium of XX-placenta or XY-placenta explants (microbial, viral and fungal contamination) is carried out according to the guidelines for monitoring the biological safety of stem cells and their derivatives.

15: The method as claimed in claim 6, wherein for control the concentrations of biologically active proteins in a conditioned medium of XY-placenta explants perfumed ELISA-test of references levels for: EGF—from 10 to 18 pg/ml; FGF-7—from 74 to 300 pg/ml; HGF—from 941 to 2039 pg/ml; TNF-alpha—from 340 to 521 pg/ml; IGF-1—from 630 to 928 ng/ml; VEGF-A—from 12 to 38 ng/ml; SCF—from 15.5 to 24 ng/ml; TGF-beta 1—from 66.5 to 178 ng/ml; ANGPT1—from 94 to 170 ng/ml; bFGF—from 58 to 97 ng/ml.

16: The method as claimed in claim 6, wherein for control the concentrations of biologically active proteins in a conditioned medium of XX-placenta explants perfumed EFISA-test of references levels for: EGF—from 8 to 18 pg/ml; FGF-7—from 90 to 265 pg/ml; HGF—from 30 to 104 pg/ml; TNF-alpha—from 340 to 546 pg/ml; IGF-1—from 514 to 681 ng/ml; VEGF-A—from 16 to 39 ng/ml; SCF—from 7.5 to 12 ng/ml; TGF-beta 1—from 311 to 550 ng/ml; ANGPT1—from 104 to 156 ng/ml; bFGF—from 14 to 43 ng/ml.

17: The method as claimed in claim 6, wherein adding of XX- or XY-placental compositions of biologically active proteins to the ingredients of cosmetic products, which are compatible with biologically active proteins and do not destroy them.

18: The method as claimed in claim 9, wherein controlled concentrations of biologically active proteins of the XY-placenta in ready-to-use cosmetic products must be not less then for: EGF—1.0 pg/ml; FGF—7-7.4 pg/ml; HGF—94.1 pg/ml; TNF-alpha—34.0 pg/ml; IGF-1—63.0 ng/ml; VEGF-A—1.2 ng/ml; SCF—1.6 ng/ml; TGF-beta 1—6.7 ng/ml; ANGPT1— 9.4 ng/ml; bFGF—5.8 ng/ml.

19: The method as claimed in claim 9, wherein controlled concentrations of biologically active proteins of the XX-placenta in ready-to-use cosmetic products must be not less then for: EGF—0.8 pg/ml; FGF—7-9.0 pg/ml; HGF—3.0 pg/ml; TNF-alpha—34.0 pg/ml; IGF-1—51.4 ng/ml; VEGF-A—1.6 ng/ml; SCF—0.8 ng/ml; TGF-beta 1—31.1 ng/ml; ANGPT1— 10.4 ng/ml; bFGF—1.4 ng/ml.
